(19) United States
(12) Patent Application Publication
(10) Pub. No.: US 2022/0055006 A1
(43) Pub. Date: Feb. 24, 2022

(54) PROCESS FOR PREPARING POLYAMIDE MICROCAPSULES

(71) Applicant: Firmenich SA, Satigny (CH)

(72) Inventors: Amal ELABBADI, Satigny (CH); Marlène JACQUEMOND, Satigny (CH); Damien BERTHIER, Satigny (CH); Lahoussine OUALI, Satigny (CH); Anaick NICOLAE, Satigny (CH)

(21) Appl. No.: 17/416,379

(22) PCT Filed: Dec. 19, 2019
(86) PCT No.: PCT/EP2019/086329

§ 371 Date: Jun. 18, 2021

(30) Foreign Application Priority Data
Dec. 19, 2018 (EP) 18214079.8

(51) Int. Cl. B01J 13/16; C08L 77/04

(52) U.S. Cl. CPC B01J 13/16; A01N 25/28; C08L 77/04

(57) ABSTRACT

Described herein is a new process for the preparation of polyamide microcapsules. Polyamide microcapsules obtainable by the process are also described. Perfuming compositions and consumer products including capsules, in particular perfumed consumer products in the form of home care or personal care products, are also described.

### TECHNICAL FIELD

The present invention relates to a new process for the preparation of polyamide microcapsules. Polyamide microcapsules obtainable by said process are also an object of the invention. Perfuming compositions and consumer products comprising said microcapsules, in particular perfumed consumer products in the form of home care or personal care products, are also part of the invention.

### BACKGROUND OF THE INVENTION

One of the problems faced by the perfumery industry lies in the relatively rapid loss of olfactive benefit provided by odoriferous compounds due to their volatility, particularly that of “top-notes”. In order to tailor the release rates of volatiles, delivery systems such as microcapsules containing a perfume are needed to protect and later release the core payload when triggered. A key requirement from the industry regarding these systems is to survive suspension in challenging bases without physically dissociating or degrading. This is referred to as stability for the delivery system. For instance, fragranced personal and household cleansers containing high levels of aggressive surfactant detergents are very challenging for the stability of microcapsules.

Polyurea and polyurethane-based microcapsule slurry are widely used for example in perfumery industry for instance as they provide a long lasting pleasant olfactory effect after their applications on different substrates. Those microcapsules have been widely disclosed in the prior art (see for example WO2007/004166 or EP 2300146 from the Applicant).

Therefore, there is still a need to provide new microcapsules, while not compromising on the performance of the microcapsules, in particular in terms of stability in a challenging medium such as a consumer product base, as well as in delivering a good performance in terms of hydrophobic material delivery, e.g. olfactive performance in the case of perfuming ingredients.

The present invention is proposing a solution to the above-mentioned problem by providing a new process for the preparation of polyamide microcapsules.

### SUMMARY OF THE INVENTION

It has now been found that performing core-shell microcapsules encapsulating hydrophobic material, preferably hydrophobic active ingredient could be obtained by reacting an acyl chloride with at least one amino-compound during the interfacial polymerization. The process of the invention therefore provides a solution to the above-mentioned problems as it allows preparing microcapsules with the desired stability in challenging bases.

In a first aspect, the present invention relates to a process for preparing a polyamide core-shell microcapsule slurry comprising the following steps:

a) Dissolving at least one acyl chloride and at least one stabilizer in a hydrophobic material, preferably a perfume to form an oil phase;
b) Dispersing the oil phase obtained in step a) into a water phase comprising optionally an amino compound A or a base to form an oil-in-water emulsion; and
c) Performing a curing step to form polyamide microcapsules in the form of a slurry.
wherein at least an amino compound B is added in the water phase before the formation of the oil-in-water emulsion and/or in the oil-in water emulsion obtained after step b)

A second object of the invention is a polyamide core-shell microcapsule slurry obtainable by the process as defined above.

A perfuming composition comprising:
(i) microcapsule slurry as defined above, wherein the hydrophobic material comprises a perfume,
(ii) at least one ingredient selected from the group consisting of a perfumery carrier and a perfumery base
(iii) optionally at least one perfumery adjuvant,
   is another object of the invention.
   Another object of the invention is a consumer product comprising:
   a personal care active base, and
   microcapsules as defined above or the perfuming composition as defined above, wherein the consumer product is in the form of a personal care composition.
      Another object of the invention is a consumer product comprising:
   a home care or a fabric care active base, and
   microcapsules as defined above or the perfuming composition as defined above, wherein the consumer product is in the form of a home care or a fabric care composition.

### DETAILED DESCRIPTION OF THE INVENTION

Unless stated otherwise, percentages (%) are meant to designate a percentage by weight of a composition.

By “active ingredient”, it is meant a single compound or a combination of ingredients.

By “perfume or flavour oil”, it is meant a single perfuming or flavouring compound or a mixture of several perfuming or flavouring compounds.

By “consumer product” or “end-product” it is meant a manufactured product ready to be distributed, sold and used by a consumer.

For the sake of clarity, by the expression “dispersion” in the present invention it is meant a system in which particles are dispersed in a continuous phase of a different composition and it specifically includes a suspension or an emulsion.

A “microcapsule”, or the similar, in the present invention, it is meant that core-shell microcapsules have a particle size distribution in the micron range (e.g. a mean diameter (d(v, 0.5)) comprised between about 1 and 3000 microns) and comprise an external solid polymer-based shell and an internal continuous oil phase enclosed by the external shell.

By “amino-compound” it should be understood a compound having at least two reactive amine groups.

In the present invention, the wordings “acyl chloride” or “acid chloride” are used indifferently.

By “polyamide microcapsules”, it means that the microcapsule's shell comprises a polyamide material. The wording “polyamide microcapsules” can also encompass a shell made of a composite comprising a polyamide material and another material, for example a biopolymer.

It has been found that polyamide core-shell microcapsules with overall good performance in terms of stability in challenging bases could be obtained when a stabilizer is added in the oil phase during the process.

### Process for Preparing a Polyamide Microcapsule Slurry

A first object of the invention is therefore a process for preparing a polyamide core-shell microcapsule slurry comprising the following steps:

a) Dissolving at least one acyl chloride and at least one stabilizer in a hydrophobic material, preferably a perfume to form an oil phase;
b) Dispersing the oil phase obtained in step a) into a water phase comprising optionally an amino compound A or a base to form an oil-in-water emulsion; and
c) Performing a curing step to form polyamide microcapsules in the form of a slurry.
wherein at least an amino compound B is added in the water phase before the formation of the oil-in-water emulsion and/or in the oil-in water emulsion obtained after step b)

According to an embodiment, the process comprises the steps of:

a) Dissolving at least one acyl chloride and at least one stabilizer in a hydrophobic material, preferably a perfume to form an oil phase;
b) Dispersing the oil phase obtained in step a) into a water phase comprising optionally an amino compound A or a base to form an oil-in-water emulsion;
c) Adding to the oil-in-water emulsion obtained in step b) an amino compound B; and
d) Performing a curing step to form polyamide microcapsules in the form of a slurry.

In one step of the process, an oil phase is formed by admixing at least one hydrophobic material with at least one acyl chloride and a stabilizer.

According to a particular embodiment, the acyl chloride is chosen in the group consisting of benzene-1,3,5-tricarbonyl chloride, benzene-1,2,4-tricarbonyl trichloride, benzene-1,2,4,5-tetracarbonyl tetrachloride, cyclohexane-1,3,5-tricarbonyl trichloride, isophthalyol dichloride, diglycolyl dichloride, succinic dichloride, and mixtures thereof.
The weight ratio between acyl chloride and the hydrophobic material is preferably comprised between 0.01 and 0.09, more preferably between 0.03 and 0.07.

The acyl chloride can be dissolved directly in the perfume oil or can be pre-dispersed in an inert solvent such as benzyl benzoate before mixing with the hydrophobic material, preferably a perfume oil.

According to a particular embodiment, a polyisocyanate having at least two isocyanate functional groups is added in the oil phase.

Suitable polyisocyanates used according to the invention include aromatic polyisocyanate, aliphatic polyisocyanate and mixtures thereof. Said polyisocyanate comprises at least 2, preferably at least 3 but may comprise up to 6, or even only 4, isocyanate functional groups. According to a particular embodiment, a triisocyanate (3 isocyanate functional group) is used.

According to one embodiment, said polyisocyanate is an aromatic polyisocyanate.

The term “aromatic polyisocyanate” is meant here as encompassing any polyisocyanate comprising an aromatic moiety. Preferably, it comprises a phenyl, a toluyl, a xylyl, a naphthyl or a diphenyl moiety, more preferably a toluyl or a xylyl moiety. Preferred aromatic polyisocyanates are biurets, polyisocyanurates and trimethylol propane adducts of diisocyanates, more preferably comprising one of the above-cited specific aromatic moieties. More preferably, the aromatic polyisocyanate is a polyisocyanurate of toluene diisocyanate (commercially available from Bayer under the tradename Desmodur® RC), a trimethylol propane-adduct of toluene diisocyanate (commercially available from Bayer under the tradename Desmodur® L75), a trimethylol propane-adduct of xylylene diisocyanate (commercially available from Mitsui Chemicals under the tradename Takenate® D-110N). In a most preferred embodiment, the aromatic polyisocyanate is a trimethylol propane-adduct of xylylene diisocyanate.

According to another embodiment, said polyisocyanate is an aliphatic polyisocyanate. The term “aliphatic polyisocyanate” is defined as a polyisocyanate which does not comprise any aromatic moiety. Preferred aliphatic polyisocyanates are a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100), among which a biuret of hexamethylene diisocyanate is even more preferred.

According to another embodiment, the at least one polyisocyanate is in the form of a mixture of at least one aliphatic polyisocyanate and of at least one aromatic polyisocyanate, both comprising at least two or three isocyanate functional groups, such as a mixture of a biuret of hexamethylene diisocyanate with a trimethylol propane-adduct of xylylene diisocyanate, a mixture of a biuret of hexamethylene diisocyanate with a polyisocyanurate of toluene diisocyanate and a mixture of a biuret of hexamethylene diisocyanate with a trimethylol propane-adduct of toluene diisocyanate. Most preferably, it is a mixture of a biuret of hexamethylene diisocyanate with a trimethylol propane-adduct of xylylene diisocyanate. Preferably, when used as a mixture the molar ratio between the aliphatic polyisocyanate and the aromatic polyisocyanate is ranging from 80:20 to 10:90.

According to an embodiment, the at least one polyisocyanate used in the process of the invention is present in amounts representing from 0.1 to 15%, preferably from 0.5 to 10% and more preferably from 0.8 to 6%, and even more preferably between 1 and 3% by weight based on the total amount of the oil phase.

### Hydrophobic Material

According to an embodiment, the hydrophobic material is a hydrophobic active ingredient. By “hydrophobic active ingredient”, it is meant any hydrophobic active ingredient—single ingredient or a mixture of ingredients—which forms a two-phase dispersion when mixed with water. The hydrophobic active ingredient is liquid at about 20° C.

Hydrophobic active ingredients are preferably chosen from the group consisting of flavor, flavor ingredients, perfume, perfume ingredients, nutraceuticals, cosmetics, pest control agents, biocide actives and mixtures thereof.

According to a particular embodiment, the hydrophobic active ingredient comprises a mixture of a perfume with another ingredient selected from the group consisting of nutraceuticals, cosmetics, pest control agents and biocide actives.

According to a particular embodiment, the hydrophobic active ingredient comprises a mixture of biocide actives with another ingredient selected from the group consisting of perfume, nutraceuticals, cosmetics, pest control agents.

According to a particular embodiment, the hydrophobic active ingredient comprises a mixture of pest control agents with another ingredient selected from the group consisting of perfume, nutraceuticals, cosmetics, biocide actives.

According to a particular embodiment, the hydrophobic active ingredient comprises a perfume.

According to a particular embodiment, the hydrophobic active ingredient consists of a perfume.

According to a particular embodiment, the hydrophobic active ingredient consists of biocide actives.

According to a particular embodiment, the hydrophobic active ingredient consists of pest control agents.

By “perfume” (or also “perfume oil”) what is meant here is an ingredient or composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odour. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes combination of perfuming ingredients with substances which together improve, enhance or modify the delivery of the perfuming ingredients, such as perfume precursors, emulsions or dispersions, as well as combinations which impart an additional benefit beyond that of modifying or imparting an odor, such as long-lasting, blooming, malodour counteraction, antimicrobial effect, microbial stability, pest control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said ingredients may also be compounds known to release in a controlled manner various types of perfuming compounds.

In particular one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0˜2,7˜]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
   Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
   Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl)propanal, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
   Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
   Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
   Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0^{2,7}]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
   Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl)butanal.

It is also understood that said ingredients may also be compounds known to release in a controlled manner various types of perfuming compounds also known as properfume or profragrance. Non-limiting examples of suitable properfume may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 2-phenylethyl oxo(phenyl)acetate or a mixture thereof.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

Preferred perfuming ingredients are those having a high steric hindrance and in particular those from one of the following groups:
Group 1: perfuming ingredients comprising a cyclohexane, cyclohexene, cyclohexanone or cyclohexenone ring substituted with at least one linear or branched C₁ to C₄ alkyl or alkenyl substituent;
Group 2: perfuming ingredients comprising a cyclopentane, cyclopentene, cyclopentanone or cyclopentenone ring substituted with at least one linear or branched C₄ to C₈ alkyl or alkenyl substituent;
Group 3: perfuming ingredients comprising a phenyl ring or perfuming ingredients comprising a cyclohexane, cyclohexene, cyclohexanone or cyclohexenone ring substituted with at least one linear or branched C₅ to C₈ alkyl or alkenyl substituent or with at least one phenyl substituent and optionally one or more linear or branched C₁ to C₃ alkyl or alkenyl substituents;
Group 4: perfuming ingredients comprising at least two fused or linked C₅ and/or C₆ rings;
Group 5: perfuming ingredients comprising a camphor-like ring structure;
Group 6: perfuming ingredients comprising at least one C7 to C20 ring structure;
Group 7: perfuming ingredients having a log P value above 3.5 and comprising at least one tert-butyl or at least one trichloromethyl substitutent;

Examples of ingredients from each of these groups are:
Group 1: 2,4-dimethyl-3-cyclohexene-1-carbaldehyde (origin: Firmenich SA, Geneva, Switzerland), isocyclocitral, menthone, isomenthone, Romascone® (methyl 2,2-dimethyl-6-methylene-1-cyclohexanecarboxylate, origin: Firmenich SA, Geneva, Switzerland), nerone, terpineol, dihydroterpineol, terpenyl acetate, dihydroterpenyl acetate, dipentene, eucalyptol, hexylate, rose oxide, Perycorolle® ((S)-1,8-p-menthadiene-7-ol, origin: Firmenich SA, Geneva, Switzerland), 1-p-menthene-4-ol, (1RS,3RS,4SR)-3-p-mentanyl acetate, (1R,2S,4R)-4,6,6-trimethyl-bicyclo[3,1,1]heptan-2-ol, Doremox® (tetrahydro-4-methyl-2-phenyl-2H-pyran, origin: Firmenich SA, Geneva, Switzerland), cyclohexyl acetate, cyclanol acetate, Fructalate® (1,4-cyclohexane diethyldicarboxylate, origin: Firmenich SA, Geneva, Switzerland), Koumalactone® ((3ARS,6SR,7ASR)-perhydro-3,6-dimethyl-benzo[B]furan-2-one, origin: Firmenich SA, Geneva, Switzerland), Natactone® ((6R)-perhydro-3,6-dimethyl-benzo[B]furan-2-one, origin: Firmenich SA, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan SA, Vernier, Switzerland), (1′R,E)-2-ethyl-4-(2′,2′,3′-trimethyl-3′-cyclopenten-1′-yl)-2-buten-1-ol (origin: Firmenich SA, Geneva, Switzerland), Polysantol® ((1′R,E)-3,3-dimethyl-5-(2′,2′,3′-trimethyl-3′-cyclopenten-1′-yl)-4-penten-2-ol, origin: Firmenich SA, Geneva, Switzerland), fleuramone, Hedione® HC (methyl-cis-3-oxo-2-pentyl-1-cyclopentane acetate, origin: Firmenich SA, Geneva, Switzerland), Veloutone® (2,2,5-Trimethyl-5-pentyl-1-cyclopentanone, origin: Firmenich SA, Geneva, Switzerland), Nirvanol® (3,3-dimethyl-5-(2,2,3-trimethyl-3-cyclopenten-1-yl)-4-penten-2-ol, origin: Firmenich SA, Geneva, Switzerland), 3-methyl-5-(2,2,3-trimethyl-3-cyclopenten-1-yl)-2-pentanol (origin, Givaudan SA, Vernier, Switzerland);
Group 3: damascones, Neobutenone® (1-(5,5-dimethyl-1-cyclohexen-1-yl)-4-penten-1-one, origin: Firmenich SA, Geneva, Switzerland), nectalactone ((1′R)-2-[2-(4′-methyl-3′-cyclohexen-1′-yl)propyl]cyclopentanone), alpha-ionone, beta-ionone, damascenone, Dynascone® (mixture of 1-(5,5-dimethyl-1-cyclohexen-1-yl)-4-penten-1-one and 1-(3,3-dimethyl-1-cyclohexen-1-yl)-4-penten-1-one, origin: Firmenich SA, Geneva, Switzerland), Dorinone® beta (1-(2,6,6-trimethyl-1-cyclohexen-1-yl)-2-buten-1-one, origin: Firmenich SA, Geneva, Switzerland), Romandolide® ((1S,1′R)-[1-(3′,3′-Dimethyl-1′-cyclohexyl)ethoxycarbonyl]methyl propanoate, origin: Firmenich SA, Geneva, Switzerland), 2-tert-butyl-1-cyclohexyl acetate (origin: International Flavors and Fragrances, USA), Limbanol® (1-(2,2,3,6-tetramethyl-cyclohexyl)-3-hexanol, origin: Firmenich SA, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, Lorysia® (4-(1,1-dimethylethyl)-1-cyclohexyl acetate, origin: Firmenich SA, Geneva, Switzerland), 8-methoxy-1-p-menthene, Helvetolide® ((1S,1′R)-2-[1-(3′,3′-dimethyl-1′-cyclohexyl) ethoxy]-2-methylpropyl propanoate, origin: Firmenich SA, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate, 2-methoxy-4-methylphenyl methyl carbonate, ethyl 2-methoxy-4-methylphenyl carbonate, 4-ethyl-2-methoxyphenyl methyl carbonate;
Group 4: Methyl cedryl ketone (origin: International Flavors and Fragrances, USA), Verdylate, vetyverol, vetyverone, 1-(octahydro-2,3,8,8-tetramethyl-2-naphtalenyl)-1-ethanone (origin: International Flavors and Fragrances, USA), (5RS,9RS,10SR)-2,6,9,10-tetramethyl-1-oxaspiro[4.5]deca-3,6-diene and the (5RS,9SR,10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), Hivernal® (a mixture of 3-(3,3-dimethyl-5-indanyl)propanal and 3-(1,1-dimethyl-5-indanyl)propanal, origin: Firmenich SA, Geneva, Switzerland), Rhubofix® (3′,4-dimethyl-tricyclo[6.2.1.0(2,7)]undec-4-ene-9-spiro-2′-oxirane, origin: Firmenich SA, Geneva, Switzerland), 9/10-ethyldiene-3-oxatricyclo[6.2.1.0(2,7)]undecane, Polywood® (perhydro-5,5,8A-trimethyl-2-naphthalenyl acetate, origin: Firmenich SA, Geneva, Switzerland), octalynol, Cetalox® (dodecahydro-3a,6,6,9a-tetramethyl-naphtho[2,1-b]furan, origin: Firmenich SA, Geneva, Switzerland), tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate, (+)-(1S,2S,3S)-2,6,6-trimethyl-bicyclo[3.1.1]heptane-3-spiro-2′-cyclohexen-4′-one;
Group 5: camphor, borneol, isobornyl acetate, 8-isopropyl-6-methyl-bicyclo[2.2.2]oct-5-ene-2-carbaldehyde, camphopinene, cedramber (8-methoxy-2,6,6,8-tetramethyl-tricyclo[5.3.1.0(1,5)]undecane, origin: Firmenich SA, Geneva, Switzerland), cedrene, cedrenol, cedrol, Florex® (mixture of 9-ethylidene-3-oxatricyclo[6.2.1.0(2,7)]undecan-4-one and 10-ethylidene-3-oxatricyclo[6.2.1.0(2,7)]undecan-4-one, origin: Firmenich SA, Geneva, Switzerland), 3-methoxy-7,7-dimethyl-10-methylene-bicyclo[4.3.1]decane (origin: Firmenich SA, Geneva, Switzerland);
Group 6: Cedroxyde® (trimethyl-13-oxabicyclo-[10.1.0]-trideca-4,8-diene, origin: Firmenich SA, Geneva, Switzerland), Ambrettolide LG ((E)-9-hexadecen-16-olide, origin: Firmenich SA, Geneva, Switzerland), Habanolide® (pentadecenolide, origin: Firmenich SA, Geneva, Switzerland), muscenone (3-methyl-(4/5)-cyclopentadecenone, origin: Firmenich SA, Geneva, Switzerland), muscone (origin: Firmenich SA, Geneva, Switzerland), Exaltolide® (pentadecanolide, origin: Firmenich SA, Geneva, Switzerland), Exaltone® (cyclopentadecanone, origin: Firmenich SA, Geneva, Switzerland), (1-ethoxyethoxy)cyclododecane (origin: Firmenich SA, Geneva, Switzerland), Astrotone, 4,8-cyclododecadien-1-one;
Group 7: Lilial® (origin: Givaudan SA, Vernier, Switzerland), rosinol.

Preferably, the perfume comprises at least 30%, preferably at least 50%, more preferably at least 60% of ingredients selected from Groups 1 to 7, as defined above. More preferably said perfume comprises at least 30%, preferably at least 50% of ingredients from Groups 3 to 7, as defined above. Most preferably said perfume comprises at least 30%, preferably at least 50% of ingredients from Groups 3, 4, 6 or 7, as defined above.

According to another preferred embodiment, the perfume comprises at least 30%, preferably at least 50%, more preferably at least 60% of ingredients having a log P above 3, preferably above 3.5 and even more preferably above 3.75.

Preferably, the perfume used in the invention contains less than 10% of its own weight of primary alcohols, less than 15% of its own weight of secondary alcohols and less than 20% of its own weight of tertiary alcohols. Advantageously, the perfume used in the invention does not contain any primary alcohols and contains less than 15% of secondary and tertiary alcohols.

According to an embodiment, the oil phase (or the oil-based core) comprises:
25-100 wt % of a perfume oil comprising at least 15 wt % of high impact perfume raw materials having a Log T<−4, and
0-75 wt % of a density balancing material having a density greater than 1.07 g/cm³.
   “High impact perfume raw materials” should be understood as perfume raw materials having a Log T<−4. The odor threshold concentration of a chemical compound is determined in part by its shape, polarity, partial charges and molecular mass. For convenience, the threshold concentration is presented as the common logarithm of the threshold concentration, i.e., Log [Threshold] (“Log T”).
   A “density balancing material” should be understood as a material having a density greater than 1.07 g/cm³ and having preferably low or no odor.
   The odor threshold concentration of a perfuming compound is determined by using a gas chromatograph (“GC”). Specifically, the gas chromatograph is calibrated to determine the exact volume of the perfume oil ingredient injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of the perfuming compound. To determine the threshold concentration, solutions are delivered to the sniff port at the back-calculated concentration. A panelist sniffs the GC effluent and identifies the retention time when odor is noticed. The average across all panelists determines the odor threshold concentration of the perfuming compound. The determination of odor threshold is described in more detail in C. Vuilleumier et al., Multidimensional Visualization of Physical and Perceptual Data Leading to a Creative Approach in Fragrance Development, Perfume & Flavorist, Vol. 33, September, 2008, pages 54-61.
   The nature of high impact perfume raw materials having a Log T<−4 and density balancing material having a density greater than 1.07 g/cm³ are described in WO2018115250, the content of which are included by reference.

According to an embodiment, the high impact perfume raw materials having a Log T<−4 are selected from the list in Table A below.

TABLE A
high impact perfume raw materials having a Log T<-4
Perfume raw materials (Log T<-4)

(+-)-1-METHOXY-3-HEXANETHIOL
4-(4-HYDROXY-1-PRENYL)-2-BUTANONE
(+-)-2-(4-METHYL-3-CYCLOHEXEN-1-YL)-2-PROPANETHIOL
2-METHOXY-4-(1-PROPENYL)-1-PRENYL ACETATE
PYRAZOBUTYLE
3-PROPYLPHENOL
1-(3-METHYL-1-BENZOFURAN-2-YL)ETHANONE
2-(3-PHENYLPROPYL)PYRIDINE
1-(3,3-DIMETHYL-1-CYCLOHEXEN-1-YL)-4-PENTEN-1-ONE (A) +
1-(5,5-DIMETHYL-1-CYCLOHEXEN-1-YL)-4-PENTEN-1-ONE
(B)
1-(5,5-DIMETHYL-1-CYCLOHEXEN-1-YL)-4-PENTEN-1-ONE
(3RS,3ARS,6SR,7ASR)-PERHYDRO-3,6-DIMETHYL-
BENZO[B]FURAN-2-ONE (A) + (3SR,3ARS,6SR,7ASR)-
PERHYDRO-3,6-DIMETHYL-BENZO[B]FURAN-2-ONE (B)
(+-)-1-(5-ETHYL-5-METHYL-1-CYCLOHEXEN-1-YL)-4-PENTEN-
1-ONE
(1′S,3′R)-1-METHYL-2-[(1′,2′,2′-TRIMETHYLBICYCLO[3.1.0]HEX-
3′-YL)METHYL]CYCLOPROPYL}METHANOL
(+-)-3-MERCAPTOHEXYL ACETATE
(2E)-1-(2,6,6-TRIMETHYL-1,3-CYCLOHEXADIEN-1-YL)-2-
BUTEN-1-ONE
7-METHYL-2H-1,5-BENZODIOXEPIN-3(4H)-ONE
(2E,6Z)-2,6-NONADIEN-1-OL
(4Z)-4-DODECENAL
(+-)-4-HYDROXY-2,5-DIMETHYL-3(2H)-FURANONE
METHYL 2,4-DIHYDROXY-3,6-DIMETHYLBENZOATE
3-METHYLINDOLE
(+-)-PERHYDRO-4ALPHA,8ABETA-DIMETHYL-4A-
NAPHTHALENOL
PATCHOULOL
2-METHOXY-4-(1-PROPENYL)PHENOL
(+-)-5,6-DIHYDRO-4-METHYL-2-PRENYL-2H-PYRAN (A) +
TETRAHYDRO-4-METHYLENE-2-PRENYL-2H-PYRAN (B)
4-METHYLENE-2-PHENYLTETRAHYDRO-2H-PYRAN (A) + (+-)-
4-METHYL-2-PHENYL-3,6-DIHYDRO-2H-PYRAN (B)
4-HYDROXY-3-METHOXYBENZALDEHYDE
NONYLENIC ALDEHYDE
2-METHOXY-4-PROPYLPHENOL
(2Z)-3-METHYL-5-PRENYL-2-PENTENENITRILE (A) + (2E)-3-
METHYL-5-PRENYL-2-PENTENENITRILE (B)
1-(SPIRO[4.5]DEC-6-EN-7-YL)-4-PENTEN-1-ONE (A) + 1-
(SPIRO[4.5]DEC-7-EN-7-YL)-4-PENTEN-1-ONE (B)
2-METHOXYNAPHTHALENE
(−)-(3AR,5A5,9A5,9BR)-3A,6,6,9A-
TETRAMETHYLDODECAHYDRONAPHTHO[2,1-B ]FURAN
5-NONANOLIDE
(3AR,5A5,9A5,9BR)-3A,6,6,9A-
TETRAMETHYLDODECAHYDRONAPHTHO[2,1-B ]FURAN
7-ISOPROPYL-2H,4H-1,5-BENZODIOXEPIN-3-ONE
COUMARIN
4-METHYLPHENYL ISOBUTYRATE
(2E)-1-(2,6,6-TRIMETHYL-1,3-CYCLOHEXADIEN-1-YL)-2-BUTEN-
1-ONE
BETA,2,2,3-TETRAMETHYL-DELTA-METHYLENE-3-
CYCLOPENTENE-1-BUTANOL
DELTA DAMASCONE ((2E)-1-[(1RS,25R)-2,6,6-TRIMETHYL-3-
CYCLOHEXEN-1-YL]-2-BUTEN-1-ONE)
(+-)-3,6-DIHYDRO-4,6-DIMETHYL-2-PRENYL-2H-PYRAN
ANISALDEHYDE
PARACRESOL
3-ETHOXY-4-HYDROXYBENZALDEHYDE
METHYL 2-AMINOBENZOATE
ETHYL METHYLPHENYLGLYCIDATE
OCTALACTONE G
ETHYL 3-PRENYL-2-PROPENOATE
(−)-(2E)-2-ETHYL-4-[(1R)-2,2,3-TRIMETHYL-3-CYCLOPENTEN-1-
YL]-2-BUTEN-1-OL
PARACRESYL ACETATE
DODECALACTONE
TRICYCLONE
(+)-(3R,5Z)-3-METHYL-5-CYCLOPENTADECEN-1-ONE
UNDECALACTONE
(1R,4R)-8-MERCAPTO-3-P-MENTHANONE
(3S,3AS,6R,7AR)-3,6-DIMETHYLHEXAHYDRO-1-BENZOFURAN-
2(3H)-ONE
BETA IONONE
(+-)-6-PENTYLTETRAHYDRO-2H-PYRAN-2-ONE
(3E,5Z)-1,3,5-UNDECATRIENE
10-UNDECENAL (A) + (9E)-9-UNDECENAL (B) + (9Z)-9-
UNDECENAL (C)
(Z)-4-DECENAL
(+-)-ETHYL 2-METHYLPENTANOATE
1,2-DIALLYLDISULFANE
(2Z)-2-TRIDECENENITRILE (A) + (3Z)-3-TRIDECENENITRILE
(B) + (3E)-3-TRIDECENENITRILE (C) + (2E)-2-
TRIDECENENITRILE (D)
(+-)-2-ETHYL-4,4-DIMETHYL-1,3-OXATHIANE
(+)-(3R,5Z)-3-METHYL-5-CYCLOPENTADECEN-1-ONE
3-(4-TERT-BUTYLPHENYL)PROPANAL
ALLYL (CYCLOHEXYLOXY)ACETATE
METHYLNAPHTHYLKETONE
(+-)-(4E)-3-METHYL-4-CYCLOPENTADECEN-1-ONE (A) + (+-)-
(5E)-3-METHYL-5-CYCLOPENTADECEN-1-ONE (B) + (+-)-(5Z)-3-
METHYL-5-CYCLOPENTADECEN-1-ONE (C)
CYCLOPROPYLMETHYL (3Z)-3-HEXENOATE (A) +
CYCLOPROPYLMETHYL (3E)-3-HEXENOATE (B)
(4E)-4-METHYL-5-(4-METHYLPHENYL)-4-PENTENAL
(+-)-1-(5-PROPYL-1,3-BENZODIOXOL-2-YL)ETHANONE
4-METHYL-2-PENTYLPYRIDINE
(+-)-(E)-3-METHYL-4-(2,6,6-TRIMETHYL-2-CYCLOHEXEN-1-
YL)-3-BUTEN-2-ONE
(3ARS,5ASR,9ASR,9BRS)-3A,6,6,9A-
TETRAMETHYLDODECAHYDRONAPHTHO[2,1-B]FURAN
(2S,5R)-5-METHYL-2-(2-PROPANYL)CYCLOHEXANONE OXIME
6-HEXYLTETRAHYDRO-2H-PYRAN-2-ONE
(+-)-3-(3-ISOPROPYL-1-PHENYL)BUTANAL
METHYL 2-((1RS,2RS)-3-OXO-2-
PENTYLCYCLOPENTYL)ACETATE (A) + METHYL 2-((1RS,2SR)-
3-OXO-2-PENTYLCYCLOPENTYL)ACETATE (B)
1-(2,6,6-TRIMETHYL-1-CYCLOHEX-2-ENYL)PENT-1-EN-3-ONE
INDOL
7-PROPYL-2H,4H-1,5-BENZODIOXEPIN-3-ONE
ETHYL PRALINE
(4-METHYLPHENOXY)ACETALDEHYDE
ETHYL TRICYCLO[5.2.1.0.(2,6)]DECANE-2-CARBOXYLATE
(+)-(1′S,2S,E)-3,3-DIMETHYL-5-(2′,2′,3′-TRIMETHYL-3′-
CYCLOPENTEN-1′-YL)-4-PENTEN-2-OL
(2R,4E)-3,3-DIMETHYL-5-[(1R)-2,2,3-TRIMETHYL-3-
CYCLOPENTEN-1-YL]-4-PENTEN-2-OL (A) + (2S,4E)-3,3-
DIMETHYL-5-[(1R)-2,2,3-TRIMETHYL-3-CYCLOPENTEN-1-YL]-
4-PENTEN-2-OL (B)
8-ISOPROPYL-6-METHYL-BICYCLO[2.2.2]OCT-5-ENE-2-
CARBALDEHYDE
METHYLNONYLACETALDEHYDE
4-FORMYL-2-METHOXYPHENYL 2-METHYLPROPANOATE
(E)-4-DECENAL
(+-)-2-ETHYL-4-(2,2,3-TRIMETHYL-3-CYCLOPENTEN-1-YL)-2-
BUTEN-1-OL
(1R,5R)-4,7,7-TRIMETHYL-6-THIABICYCLO[3.2.1]OCT-3-ENE (A) +
(1R,4R,5R)-4,7,7-TRIMETHYL-6-THIABICYCLO[3.2.1]OCTANE
(B)
(−)-(3R)-3,7-DIMETHYL-1,6-OCTADIEN-3-OL
(E)-3-PHENYL-2-PROPENENITRILE
4-METHOXYBENZYL ACETATE
(E)-3-METHYL-5-(2,2,3-TRIMETHYL-3-CYCLOPENTEN-1-YL)-4-
PENTEN-2-OL
ALLYL (3-METHYLBUTOXY)ACETATE (A) + (+-)-ALLYL (2-
METHYLBUTOXY)ACETATE
(+-)-(2E)-1-(2,6,6-TRIMETHYL-2-CYCLOHEXEN-1-YL)-2-BUTEN-
1-ONE
(1E)-1-(2,6,6-TRIMETHYL-1-CYCLOHEXEN-1-YL)-1-PENTEN-3-
ONE

According to an embodiment, perfume raw materials having a Log T<−4 are chosen in the group consisting of aldehydes, ketones, alcohols, phenols, esters lactones, ethers, epoxydes, nitriles and mixtures thereof.

According to an embodiment, perfume raw materials having a Log T<−4 comprise at least one compound chosen in the group consisting of alcohols, phenols, esters lactones, ethers, epoxydes, nitriles and mixtures thereof, preferably in amount comprised between 20 and 70% by weight based on the total weight of the perfume raw materials having a Log T<−4.

According to an embodiment, perfume raw materials having a Log T<−4 comprise between 20 and 70% by weight of aldehydes, ketones, and mixtures thereof based on the total weight of the perfume raw materials having a Log T<−4.

The remaining perfume raw materials contained in the oil-based core may have therefore a Log T>−4.

Non limiting examples of perfume raw materials having a Log T>−4 are listed in table B below.

TABLE B
perfume raw materials having a Log T>-4
Perfume raw materials (Log T>-4)

ETHYL 2-METHYLBUTYRATE
(E)-3-PRENYL-2-PROPENYL
ACETATE
(+)-8-SEC-BUTYLQUINOLINE (A) +
(+-)-6-SEC-BUTYLQUINOLINE
(+-)-3-(1,3-BENZODIOXOL-5-YL)-2-
METHYLPROPANAL
VERDYLE PROPIONATE
1-(OCTAHYDRO-2,3,8,8-
TETRAMETHYL-2-NAPHTALENYL)-
1-ETHANONE
METHYL 2-((1RS,2R5)-3-OXO-2-
PENTYLCYCLOPENTYL)ACETATE
(+-)-(E)-4-METHYL-3-DECEN-5-OL
2,4-DIMETHYL-3-CYCLOHEXENE-1-
CARBALDEHYDE
1,3,3-TRIMETHYL-2-
OXABICYCLO[2.2.2]OCTANE
TETRAHYDRO-4-METHYL-2-(2-
METHYL-1-PROPENYL)-2H-PYRAN
ALDEHYDE C 12
1-OXA-12-CYCLOHEXADECEN-2-
ONE (A) + 1-OXA-13-
CYCLOHEXADECEN-2-ONE(B)
(+-)-3-(4-ISOPROPYLPHENYL)-2-
METHYLPROPANAL
ALDEHYDE C 11 LENIQUE
(+-)-2,6-DIMETHYL-7-OCTEN-2-OL
(+-)-2,6-DIMETHYL-7-OCTEN-2-OL
ALLYL 3-
CYCLOHEXYLPROPANOATE
(Z)-3-HEXENYL ACETATE
(2RS,5SR)-5-METHYL-2-(2-
PROPANYL)CYCLOHEXANONE(A) +
(2RS,5RS)-5-METHYL-2-(2-
PROPANYL)CYCLOHEXANONE (B)
ALLYL HEPTANOATE
(1RS,2RS)-2-(2-METHYL-2-
PROPANYL)CYCLOHEXYL ACETATE
(A) + (1RS,2SR)-2-(2-METHYL-2-
PROPANYL)CYCLOHEXYL ACETATE
(B)
1,1-DIMETHYL-2-PHENYLETHYL
BUTYRATE
GERANYLACETATE (A) + NERYL
ACETATE (B)
(+-)-1-PHENYLETHYL ACETATE
1,1-DIMETHYL-2-PHENYLETHYL
ACETATE
3-METHYL-2-BUTENYL ACETATE
ETHYL 3-OXOBUTANOATE (A) <=>
(2Z)-ETHYL 3-HYDROXY-2-
BUTENOATE (B)
8-P-MENTHANOL
8-P-MENTHANYL ACETATE (A) + 1-
P-MENTHANYL ACETATE (B)
(+-)-2-(4-METHYL-3-CYCLOHEXEN-1-
YL)-2-PROPANYL ACETATE
(+-)-2-METHYLBUTYL BUTANOATE
2-{(1S)-1-[(1R)-3,3-
DIMETHYLCYCLOHEXYL]ETHOXY}-
2-OXOETHYL PROPIONATE
3,5,6-TRIMETHYL-3-CYCLOHEXENE-
1-CARBALDEHYDE(A) + 2,4,6-
TRIMETHYL-3-CYCLOHEXENE-1-
CARBALDEHYDE (B)
2-CYCLOHEXYLETHYL ACETATE
ALDEHYDE C 8
ETHYL BUTANOATE
(+-)-(3E)-4-(2,6,6-TRIMETHYL-2-
CYCLOHEXEN-1-YL)-3-BUTEN-2-
ONE(A) + (3E)-4-(2,6,6-TRIMETHYL-
1-CYCLOHEXEN-1-YL)-3-BUTEN-2-
ONE (B);
1-[(1RS,6SR)-2,2,6-
TRIMETHYLCYCLOHEXYL]-3-
HEXANOL
1,3,3-TRIMETHYL-2-
OXABICYCLO[2.2.2]OCTANE
1,3,3-TRIMETHYL-2-
OXABICYCLO[2.2.2]OCTANE
ETHYL HEXANOATE
UNDECANAL
ALDEHYDE C 10
2-PHENYLETHYL ACETATE
(1S,2S,4S)-1,7,7-
TRIMETHYLBICYCLO[2.2.1]HEPTAN-
2-OL (A) + (1S,2R,4S)-1,7,7-
TRIMETHYLBICYCLO[2.2.1]HEPTAN-
2-OL (B)
(+-)-3,7-DIMETHYL-3-OCTANOL
1-METHYL-4-(2-
PROPANYLIDENE)CYCLOHEXENE
(+)-(R)-4-(2-METHOXYPROPAN-2-
YL)-1-METHYLCYCLOHEX-1-ENE
VERDYL ACETATE
(3R)-1-[(1R,6S)-2,2,6-
TRIMETHYLCYCLOHEXYL]-3-
HEXANOL(A) + (3S)-1-[(1R,6S)-2,2,6-
TRIMETHYLCYCLOHEXYL]-3-
HEXANOL (B) + (3R)-1-[(1S,6S)-2,2,6-
TRIMETHYLCYCLOHEXYL]-3-
HEXANOL (C)
(+)-(1S,1′R)-2-[1-(3′,3′-DIMETHYL-1′-
CYCLOHEXYL)ETHOXY]-2-
METHYLPROPYL PROPANOATE

According to an embodiment, the oil phase (or the oil-based core) comprises 2-75 wt % of a density balancing material having a density greater than 1.07 g/cm³ and 25-98 wt % of a perfume oil comprising at least 15 wt % of high impact perfume raw materials having a Log T<−4.

The density of a component is defined as the ratio between its mass and its volume (g/cm³).

Several methods are available to determine the density of a component.

One may refer for example to the ISO 298:1998 method to measure d20 densities of essential oils.

According to an embodiment, the density balancing material is chosen in the group consisting of benzyl salicylate, benzyl benzoate, cyclohexyl salicylate, benzyl phenylacetate, phenylethyl phenoxyacetate, triacetin, methyl and ethyl salicylate, benzyl cinnamate, and mixtures thereof.

According to a particular embodiment, the density balancing material is chosen in the group consisting of benzyl salicylate, benzyl benzoate, cyclohexyl salicylate and mixtures thereof.

According to a particular embodiment, the hydrophobic material is free of any active ingredient (such as perfume). According to this particular embodiment, it comprises, preferably consists of hydrophobic solvents, preferably chosen in the group consisting of isopropyl myristate, tryglycerides (e.g. Neobee® MCT oil, vegetable oils), D-limonene, silicone oil, mineral oil, and mixtures thereof with optionally hydrophilic solvents preferably chosen in the group consisting of 1,4 butanediol, benzyl alcohol, triethyl citrate, triacetin, benzyl acetate, ethyl acetate, propylene glycol (1,2-propanediol), 1,3-Propanediol, dipropylene glycol, glycerol, glycol ethers and mixtures thereof.

The term “biocide” refers to a chemical substance capable of killing living organisms (e.g. microorganisms) or reducing or preventing their growth and/or accumulation. Biocides are commonly used in medicine, agriculture, forestry, and in industry where they prevent the fouling of, for example, water, agricultural products including seed, and oil pipelines. A biocide can be a pesticide, including a fungicide, herbicide, insecticide, algicide, molluscicide, miticide and rodenticide; and/or an antimicrobial such as a germicide, antibiotic, antibacterial, antiviral, antifungal, antiprotozoal and/or antiparasite.

As used herein, a “pest control agent” indicates a substance that serves to repel or attract pests, to decrease, inhibit or promote their growth, development or their activity. Pests refer to any living organism, whether animal, plant or fungus, which is invasive or troublesome to plants or animals, pests include insects notably arthropods, mites, spiders, fungi, weeds, bacteria and other microorganisms.

By “flavour ingredient or composition” it is meant here a flavouring ingredient or a mixture of flavouring ingredients, solvent or adjuvants of current use for the preparation of a flavouring formulation, i.e. a particular mixture of ingredients which is intended to be added to an edible composition or chewable product to impart, improve or modify its organoleptic properties, in particular its flavour and/or taste. Taste modulator as also encompassed in said definition. Flavouring ingredients are well known to a skilled person in the art and their nature does not warrant a detailed description here, which in any case would not be exhaustive, the skilled flavourist being able to select them on the basis of his general knowledge and according to the intended use or application and the organoleptic effect it is desired to achieve. Many of these flavouring ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor Ingredients, 1975, CRC Press or Synthetic Food Adjuncts, 1947, by M. B. Jacobs, can Nostrand Co., Inc. Solvents and adjuvants or current use for the preparation of a flavouring formulation are also well known in the art.

In a particular embodiment, the flavour is selected from the group consisting of terpenic flavours including citrus and mint oil, and sulfury flavours.

According to any one of the invention's embodiments, the hydrophobic material represents between about 10% and 60% w/w, or even between 15% and 45% w/w, by weight, relative to the total weight of the dispersion as obtained after step b).

According to a particular embodiment, the oil phase essentially consists of the acyl chloride, a perfume or flavor oil and a stabilizer.

According to the invention, a stabilizer is added in the oil phase to form later on the emulsion. According to an embodiment, the stabilizer is a colloidal stabilizer.

The colloidal stabilizer can be a molecular emulsifier (standard emulsion) or solid particles (Pickering emulsion).

By “stabilizer”, it is meant compounds capable to stabilize oil/water interface as an emulsion. According to a particular embodiment, the stabilizer is a biopolymer.

By “biopolymers” it is meant biomacromolecules produced by living organisms. Biopolymers are characterized by molecular weight distributions ranging from 1,000 (1 thousand) to 1,000,000,000 (1 billion) Daltons. These macromolecules may be carbohydrates (sugar based) or proteins (amino-acid based) or a combination of both (gums) and can be linear or branched.

According to an embodiment, the stabilizer is chosen in the group consisting of gum Arabic, modified starch, polyvinyl alcohol, PVP (polyvinylpyrolidone), CMC (carboxymethylcellulose), anionic polysaccharides, acrylamide copolymer, inorganic particles, protein such as soy protein, rice protein, whey protein, white egg albumin, sodium caseinate, gelatin, bovine serum albumin, hydrolyzed soy protein, hydrolyzed sericin, Pseudocollagen, Silk protein, sericin powder, and mixtures thereof.

According to a particular embodiment, the stabilizer is a biopolymer chosen in the group consisting of consisting of protein such as whey protein, sodium caseinate, bovine serum albumin, and mixtures thereof.

When added in the oil phase, the stabilizer can be pre-dispersed in an inert solvent such as benzyl benzoate or can be mixed to the active ingredient, preferably comprising a perfume oil.

The stabilizer and acyl chloride can be premixed and can be heated at a temperature between for example 10 and 80° C. before mixing with the hydrophobic material, preferably comprising a perfume oil.

According to any one of the above embodiments of the present invention, the dispersion comprises between about 0.01% and 3.0% of at least stabilizer, percentage being expressed on a w/w basis relative to the total weight of the dispersion as obtained after step b). In still another aspect of the invention, the dispersion comprises between about 0.05% and 2.0%, preferably between about 0.05% and 1.0% of at least a colloid stabilizer. In still another aspect of the invention, the dispersion comprises between about 0.1% and 1.6%, preferably between about 0.1% and 0.8% of at least a colloid stabilizer.

In another step of the process according to the invention, the oil phase of step a) is dispersed into an aqueous solution comprising optionally an amino compound A or a base to form an oil-in-water emulsion.

The mean droplet size of the emulsion is preferably comprised between 1 and 1000 microns, more preferably between 1 and 500 microns, and even more preferably between 5 and 50 microns.

When added, the amino compound A may be an amino-acid, preferably chosen in the group consisting of L-Lysine, L-Arginine, L-Histidine, L-Tryptophane, L-Serin, L-Glutamine, L-Threonine and mixtures thereof, preferably L-Lysine, L-Arginine, L-Histidine, L-Tryptophane and mixtures thereof, more preferably L-Lysine, L-Arginine, L-Histidine and mixtures thereof. The amino-acid has preferably two nucleophilic groups.

When added, the amino compound A may be chosen in the group consisting of L-Lysine, L-Lysine ethyl ester, guanidine carbonate, chitosan, 3-aminopropyltriethoxysilane, and mixtures thereof. According to a particular embodiment, the amino compound A is L-Lysine.

According to a particular embodiment, when added, the base is not an amino-compound and is chosen in the group consisting of sodium carbonate, sodium bicarbonate, sodium hydroxide and mixtures thereof.

Base is preferably added in an amount comprised between 0.1% and 10% by weight based on the dispersion, more preferably between 0.5% and 5% by weight.

In another step of the process according to an embodiment, an amino compound B is added to the oil-in-water emulsion obtained in step b) to form a polyamide shell.

As non-limiting examples, the amino-compound B is chosen in the group consisting of a xylylene diamine, 1,2-diaminocyclohexane, 1,4-diaminocyclohexane, L-lysine, L-Lysine ethyl ester, Jeffamine® (Polyetheramines), ethylene diamine, diethylene triamine, spermine, spermidine, polyamidoamine (PAMAM), guanidine carbonate, chitosan, tris-(2-aminoethyl)amine, 3-aminopropyltriethoxysilane, L-arginine, an amine having a disulfide bond such as cystamine, cystamine hydrochloride, cystine, cystine hydrochloride, cystine dialkyl ester, cystine dialkyl ester hydrochloride and mixtures thereof.

According to an embodiment, the amino-compound B is an amine having a disulfide bond and is chosen in the group consisting of cystamine, cystamine hydrochloride, cystine, cystine hydrochloride, cystine dialkyl ester, cystine dialkyl ester hydrochloride and mixtures thereof.

According to another embodiment, the amino-compound B is chosen in the group consisting of xylylene diamine, 1,2-diaminocyclohexane, 1,4-diaminocyclohexane, L-lysine, L-Lysine ethyl ester, Jeffamine® (Polyetheramines), ethylene diamine, diethylene triamine, spermine, spermidine, polyamidoamine (PAMAM), guanidine carbonate, chitosan, tris-(2-aminoethyl)amine, 3-aminopropyltriethoxysilane, L-arginine and mixtures thereof.

According to a particular embodiment, when amino compound A is present, the amino compound A and the amino compound B are the same.

According to another particular embodiment, when amino compound A is present, the amino compound A and the amino compound B are different.

According to an embodiment, when amino compound A is present, the weight ratio between the amino compound A and the amino compound B is comprised between 0.5 and 25, preferably between 1.3 and 10, more preferably between 1.3 and 7.

The amount of the amino compound B used is typically adjusted so that the molar ratio between the functional group NH₂ of the amino compound B and COCl of the acyl chloride is comprised between 0.01 and 7.5, preferably from 0.1 to 3.0.

When added, the amount of the amino compound A used is typically adjusted so that the molar ratio between the functional groups NH₂ of the amino compound A and the functional groups COCl of the acyl chloride is comprised between 0.2 and 3, preferably from 0.5 to 2.

According to an embodiment, a base is added to adjust the pH at the end of step c) or d). One may cite as non-limiting examples guanidine carbonate, sodium bicarbonate or triethanolamine.

Base is preferably added in an amount comprised between 0.1% and 10% based on the dispersion, more preferably between 0.5% and 5%.

This is followed by a curing step c) or d) which allows ending up with microcapsules in the form of a slurry. According to a preferred embodiment, to enhance the kinetics, said step is performed at a temperature comprised between 5 and 90° C., possibly under pressure, for 1 to 8 hours. More preferably it is performed at between 10 and 80° C. for between 30 minutes and 5 hours.

According to a particular embodiment, no polyol is added at any step of the process.

Optional outer coating: According to a particular embodiment of the invention, during or at the end of step c) or d) one may also add to the invention's slurry a polymer selected from the group consisting of a non-ionic polysaccharide, a cationic polymer and mixtures thereof to form an outer coating to the microcapsule.

Non-ionic polysaccharide polymers are well known to a person skilled in the art and are described for instance in WO2012/007438 page 29, lines 1 to 25 and in WO2013/026657 page 2, lines 12 to 19 and page 4, lines 3 to 12. Preferred non-ionic polysaccharides are selected from the group consisting of locust bean gum, xyloglucan, guar gum, hydroxypropyl guar, hydroxypropyl cellulose and hydroxypropyl methyl cellulose.

Cationic polymers are well known to a person skilled in the art. Preferred cationic polymers have cationic charge densities of at least 0.5 meq/g, more preferably at least about 1.5 meq/g, but also preferably less than about 7 meq/g, more preferably less than about 6.2 meq/g. The cationic charge density of the cationic polymers may be determined by the Kjeldahl method as described in the US Pharmacopoeia under chemical tests for Nitrogen determination. The preferred cationic polymers are chosen from those that contain units comprising primary, secondary, tertiary and/or quaternary amine groups that can either form part of the main polymer chain or can be borne by a side substituent directly connected thereto. The weight average (Mw) molecular weight of the cationic polymer is preferably between 10,000 and 3.5M Dalton, more preferably between 50,000 and 1.5M Dalton. According to a particular embodiment, one will use cationic polymers based on acrylamide, methacrylamide, N-vinylpyrrolidone, quaternized N,N-dimethylaminomethacrylate, diallyldimethylammonium chloride, quaternized vinylimidazole (3-methyl-1-vinyl-1H-imidazol-3-ium chloride), vinylpyrrolidone, acrylamidopropyltrimonium chloride, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. Preferably copolymers shall be selected from the group consisting of polyquaternium-5, polyquaternium-6, polyquaternium-7, polyquaternium 10, polyquaternium-11, polyquaternium-16, polyquaternium-22, polyquaternium-28, polyquaternium-43, polyquaternium-44, polyquaternium-46, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. As specific examples of commercially available products, one may cite Salcare® SC60 (cationic copolymer of acrylamidopropyltrimonium chloride and acrylamide, origin: BASF) or Luviquat®, such as the PQ 11N, FC 550 or Style (polyquaternium-11 to 68 or quaternized copolymers of vinylpyrrolidone origin: BASF), or also the Jaguar® (C13S or C17, origin Rhodia).

According to any one of the above embodiments of the invention, there is added an amount of polymer described above comprised between about 0% and 5% w/w, or even between about 0.1% and 2% w/w, percentage being expressed on a w/w basis relative to the total weight of the slurry as obtained after step c) or d). It is clearly understood by a person skilled in the art that only part of said added polymers will be incorporated into/deposited on the microcapsule shell.

Another object of the invention is a process for preparing a microcapsule powder comprising the steps as defined above and an additional step d) or e) consisting of submitting the slurry obtained in step c) or d) to a drying, like spray-drying, to provide the microcapsules as such, i.e. in a powdery form. It is understood that any standard method known by a person skilled in the art to perform such drying is also applicable. In particular the slurry may be spray-dried preferably in the presence of a polymeric carrier material such as polyvinyl acetate, polyvinyl alcohol, dextrins, natural or modified starch, vegetable gums, pectins, xanthans, alginates, carragenans or cellulose derivatives to provide microcapsules in a powder form.

According to a particular embodiment, the carrier material contains free perfume oil which can be the same or different from the perfume from the core of the microcapsules.

Another object of the invention is a polyamide microcapsule slurry obtainable by the process as described above. Another object of the invention is a polyamide microcapsule powder obtained by drying the microcapsule slurry defined above.

### Perfuming Composition/Consumer Products

The microcapsules of the invention can be used in combination with active ingredients. An object of the invention is therefore a composition comprising:

(i) microcapsules as defined above;

(ii) an active ingredient, preferably chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.

The microcapsules of the invention can be used for the preparation of perfuming or flavouring compositions which are also an object of the invention.

The capsules of the invention show very good performance in terms of stability in challenging medium.

Another object of the present invention is a perfuming composition comprising:

(i) microcapsules as defined above, wherein the oil comprises a perfume;

(ii) at least one ingredient selected from the group consisting of a perfumery carrier, a perfumery co-ingredient and mixtures thereof;

(iii) optionally at least one perfumery adjuvant.

As liquid perfumery carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery co-ingredient, other suitable perfumery carriers than those previously specified, can be also ethanol, water/ethanol mixtures, limonene or other terpenes, isoparaffins such as those known under the trademark Isopar© (origin: Exxon Chemical) or glycol ethers and glycol ether esters such as those known under the trademark Dowanol® (origin: Dow Chemical Company). By “perfumery co-ingredient” it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to at least impart or modify in a positive or pleasant way the odor of a composition, and not just as having an odor.

The nature and type of the perfuming co-ingredients present in the perfuming composition do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said co-ingredients may also be compounds known to release in a controlled manner various types of perfuming compounds.

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

Preferably, the perfuming composition according to the invention comprises between 0.01 and 30% by weight of microcapsules as defined above.

The invention's microcapsules can advantageously be used in many application fields and used in consumer products. Microcapsules can be used in liquid form applicable to liquid consumer products as well as in powder form, applicable to powder consumer products.

According to a particular embodiment, the consumer product as defined above is liquid and comprises:
a) from 2 to 65% by weight, relative to the total weight of the consumer product, of at least one surfactant;
b) water or a water-miscible hydrophilic organic solvent; and
c) microcapsule slurry as defined above,
d) optionally non-encapsulated perfume.

According to a particular embodiment, the consumer product as defined above is in a powder form and comprises:
a) from 2 to 65% by weight, relative to the total weight of the consumer product, of at least one surfactant;
b) microcapsule powder as defined above.
c) optionally perfume powder that is different from the microcapsules defined above.

In the case of microcapsules including a perfume oil-based core, the products of the invention, can in particular be of used in perfumed consumer products such as product belonging to fine fragrance or “functional” perfumery. Functional perfumery includes in particular personal-care products including hair-care, body cleansing, skin care, hygiene-care as well as home-care products including laundry care and air care. Consequently, another object of the present invention consists of a perfumed consumer product comprising as a perfuming ingredient, the microcapsules defined above or a perfuming composition as defined above. The perfume element of said consumer product can be a combination of perfume microcapsules as defined above and free or non-encapsulated perfume, as well as other types of perfume microcapsule than those here-disclosed.

In particular a liquid consumer product comprising:
a) from 2 to 65% by weight, relative to the total weight of the consumer product, of at least one surfactant;
b) water or a water-miscible hydrophilic organic solvent; and
c) a perfuming composition as defined above is another object of the invention.

Also a powder consumer product comprising

(a) from 2 to 65% by weight, relative to the total weight of the consumer product, of at least one surfactant; and
(b) a perfuming composition as defined above is part of the invention.

The invention's microcapsules can therefore be added as such or as part of an invention's perfuming composition in a perfumed consumer product.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumed consumer product can be a perfume, such as a fine perfume, a cologne, an after-shave lotion, a body-splash; a fabric care product, such as a liquid or solid detergent, tablets and pods, a fabric softener, a dryer sheet, a fabric refresher, an ironing water, or a bleach; a personal-care product, such as a hair-care product (e.g. a shampoo, hair conditioner, a colouring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); an air care product, such as an air freshener or a “ready to use” powdered air freshener; or a home care product, such all-purpose cleaners, liquid or power or tablet dishwashing products, toilet cleaners or products for cleaning various surfaces, for example sprays & wipes intended for the treatment/refreshment of textiles or hard surfaces (floors, tiles, stone-floors etc.); a hygiene product such as sanitary napkins, diapers, toilet paper.

Another object of the invention is a consumer product comprising:

a personal care active base, and
microcapsules slurry or microcapsule powder as defined above or the perfuming composition as defined above,
wherein the consumer product is in the form of a personal care composition.

Personal care active base in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The personal care composition is preferably chosen in the group consisting of a hair-care product (e.g. a shampoo, hair conditioner, a colouring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product).

Another object of the invention is a consumer product comprising:

a home care or a fabric care active base, and
microcapsules slurry or microcapsule powder as defined above or the perfuming composition as defined above,
wherein the consumer product is in the form of a home care or a fabric care composition.

Home care or fabric care active base in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Preferably, the consumer product comprises from 0.1 to 15 wt %, more preferably between 0.2 and 5 wt % of the microcapsules of the present invention, these percentages being defined by weight relative to the total weight of the consumer product. Of course the above concentrations may be adapted according to the benefit effect desired in each product.

According to a particular embodiment, the consumer product in which the microcapsules are incorporated has a pH preferably lower than 4.5.

### Fabric Softener

An object of the invention is a consumer product in the form of a fabric softener composition comprising:
a fabric softener active base; preferably chosen in the group consisting of dialkyl quaternary ammonium salts, dialkyl ester quaternary ammonium salts (esterquats), Hamburg esterquat (HEQ), TEAQ (triethanolamine quat), cationic guars, silicones and mixtures thereof, preferably in an amount comprised between 85 and 99.95% by weight based on the total weight of the composition,
a microcapsule slurry as defined above, preferably in an amount comprised between 0.05 to 15 wt %, more preferably between 0.1 and 5 wt % by weight based on the total weight of the composition.

### Liquid Detergent

An object of the invention is a consumer product in the form of a liquid detergent composition comprising:
a liquid detergent active base; preferably chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, preferably in an amount comprised between 85 and 99.95% by weight based on the total weight of the composition,
a microcapsule slurry as defined above, preferably in an amount comprised between 0.05 to 15 wt %, more preferably between 0.1 and 5 wt % by weight based on the total weight of the composition.

### Solid Detergent

An object of the invention is a consumer product in the form of a solid detergent composition comprising:
a solid detergent active base; preferably chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, preferably in an amount comprised between 85 and 99.95% by weight based on the total weight of the composition,
a microcapsule slurry or microcapsule powder as defined above, preferably in an amount comprised between 0.05 to 15 wt %, more preferably between 0.1 and 5 wt % by weight based on the total weight of the composition.

### Solid Scent Booster

An object of the invention is a consumer product in the form of a solid scent booster comprising:
a solid carrier, preferably chosen in the group consisting of urea, sodium chloride, sodium sulphate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, saccharides such as sucrose, mono-, di-, and polysaccharides and derivatives such as starch, cellulose, methyl cellulose, ethyl cellulose, propyl cellulose, polyols/sugar alcohols such as sorbitol, maltitol, xylitol, erythritol, and isomalt, PEG, PVP, citric acid or any water soluble solid acid, fatty alcohols or fatty acids and mixtures thereof.
microcapsules powder as defined above, in a powdered form, preferably in an amount comprised between 0.05 to 15 wt %, more preferably between 0.1 and 5 wt % by weight based on the total weight of the composition.

### Liquid Scent Booster

An object of the invention is a consumer product in the form of a liquid scent booster comprising:
an aqueous phase,
a surfactant system essentially consisting of one or more than one non-ionic surfactant, wherein the surfactant system has a mean HLB between 10 and 14, preferably chosen in the group consisting of ethoxylated aliphatic alcohols, POE/PPG (polyoxyethylene and polyoxypropylene) ethers, mono and polyglyceryl esters, sucrose ester compounds, polyoxyethylene hydroxylesters, alkyl polyglucosides, amine oxides and combinations thereof;
a linker chosen in the group consisting of alcohols, salts and esters of carboxylic acids, salts and esters of hydroxyl carboxylic acids, fatty acids, fatty acid salts, glycerol fatty acids, surfactant having an HLB less than 10 and mixtures thereof, and
microcapsules slurry as defined above, in the form of a slurry, preferably in an amount comprised between 0.05 to 15 wt %, more preferably between 0.1 and 5 wt % by weight based on the total weight of the composition.

### Shampoo/Shower Gel

An object of the invention is a consumer product in the form of a shampoo or a shower gel composition comprising:
a shampoo or a shower gel active base; preferably chosen in the group consisting of sodium alkylether sulfate, ammonium alkylether sulfates, alkylamphoacetate, cocamidopropyl betaine, cocamide MEA, alkylglucosides and aminoacid based surfactants and mixtures thereof, preferably in an amount comprised between 85 and 99.95% by weight based on the total weight of the composition,
a microcapsule slurry as defined above, preferably in an amount comprised between 0.05 to 15 wt %, more preferably between 0.1 and 5 wt % by weight based on the total weight of the composition.

### Rinse-Off Conditioner

An object of the invention is a consumer product in the form of a rinse-off conditioner composition comprising:
a rinse-off conditioner active base; preferably chosen in the group consisting of cetyltrimonium chloride, stearyl trimonium chloride, benzalkonium chloride, behentrimonium chloride and mixture thereof, preferably in an amount comprised between 85 and 99.95% by weight based on the total weight of the composition,
a microcapsule slurry as defined above, preferably in an amount comprised between 0.05 to 15 wt %, more preferably between 0.1 and 5 wt % by weight based on the total weight of the composition.

### Hair Coloration

An object of the invention is a consumer product in the form of an oxidative hair coloring composition comprising:
an oxidizing phase comprising an oxidizing agent and an alkaline phase comprising an alkakine agent, a dye precursor and a coupling compound; wherein said dye precursor and said coupling compound form an oxidative hair dye in the presence of the oxidizing agent, preferably in an amount comprised between 85 and 99.95% by weight based on the total weight of the composition,
a microcapsule slurry as defined above, preferably in an amount comprised between 0.05 to 15 wt %, more preferably between 0.1 and 5 wt % by weight based on the total weight of the composition.

By “oxidative hair coloring composition”, it is meant a composition comprising two groups of colorless dye molecules: the dye precursor and the coupling agent. Upon reaction with each other through an oxidation process, they form a wide range of colored molecules (dyes) that are then trapped into the hair due their size. In other words, the dye precursor and the coupling compound form an oxidative hair dye in the presence of the oxidizing agent.

“Dye precursor” and “oxidative dye precursor” are used indifferently in the present invention.

Dye precursors can be aromatic compounds derived from benzene substituted by at least two electron donor groups such as NH₂ and OH in para or ortho positions to confer the property of easy oxidation.

According to an embodiment, dye precursors are chosen in the group consisting of p-phenylene diamine, 2,5-diamino toluene, N,N-bis(2-hydroxymethyl)-p-phenylene diamine, 4-aminophenol, 1,4-diamino-benzene, and mixtures thereof.

The primary dye precursors is used in combination with coupling agents. Coupling agents are preferably aromatic compounds derived from benzene and substituted by groups such as NH₂ and OH in the meta position and do not produce color singly, but which modify the color, shade or intensity of the colors developed by the dye precursor.

According to an embodiment, the coupling agent is chosen in the group consisting of resorcinol, 2-methyl resorcinol, 4-chlororesorchinol, 2,5-diamino-toluene, 1,3-diamino-benzene, 2,4-diaminophenoxyethanol HCl, 2-amino-hydroxyethylaminoanisole sulfate, 4-amino-2-hydroxytoluene, and mixtures thereof.

The oxidative dye precursor is preferably used in an amount comprised between 0.001% and 5%, preferably between 0.1% and 4% by weight based on the total weight of the composition.

The use of oxidative dye precursors and coupling agents in hair coloring formulation have been widely disclosed in the prior art and is well-known from the person skilled in the art. One may cite for example EP0946133A1, the content of which is incorporated by reference.

The alkaline phase comprises an alkaline agent, preferably chosen in the group consisting of ammonia hydroxide, ammonia carbonate, ethanolamine, potassium hydroxide, sodium borate, sodium carbonate, triethanolamine and mixtures thereof.

The alkaline agent is preferably used in an amount comprised between 1% and 10%, preferably between 3% and 9% by weight based on the total weight of the composition.

According to the invention, the coupling agent and the dye precursor in an alkaline medium form an oxidative hair dye in the presence of the oxidizing agent.

The oxidizing agent will supply the necessary oxygen gas to develop color molecules and create a change in hair color.

The oxidizing agent should be safe and effective for use in the compositions herein.

Preferably, the oxidizing agents suitable for use herein will be soluble in the compositions according to the present invention when in liquid form and/or in the form intended to be used.

Preferably, oxidizing agents suitable for use herein will be water-soluble. Suitable oxidizing agents for use herein are selected from inorganic peroxygen oxidizing agents, preformed organic peroxyacid oxidizing agents and organic peroxide oxidizing agents or mixtures thereof.

The oxidizing agent is preferably used in an amount comprised between 5 and 30%, preferably between 5 and 25% by weight based on the total weight of the composition.

Components commonly used in cosmetic compositions may be added into the hair coloring composition as defined in the present invention. One may cite for example, surfactants, cationic polymers, oily substances, silicone derivatives, free perfume, preservatives, ultraviolet absorbents, antioxidants, germicides, propellants, thickeners.

According to a particular embodiment, the hair coloring composition comprises one or more quaternary ammonium compounds, preferably chosen in the group consisting of cetyltrimonium chloride, stearyl trimonium chloride, benzalkonium chloride, behentrimonium chloride and mixture thereof to confer hair conditioner benefits.

### Perfuming Composition

According to a particular embodiment, the consumer product is in the form of a perfuming composition comprising:
0.1 to 30%, preferably 0.1 to 20% of microcapsules as defined previously,
0 to 40%, preferably 3-40% of perfume, and
20-90, preferably 40-90% of ethanol, by weight based on the total weight of the perfuming composition.

The invention will now be further described by way of examples. It will be appreciated that the invention as claimed is not intended to be limited in any way by these examples.

### EXAMPLES

### General Protocol

1,3,5-benzene tricarbonyle chloride (BTC, 1.77 g, Table 1) was dissolved in benzyl benzoate (5 g). An emulsifier (0.95 g) was dispersed in benzyl benzoate (5 g) and was optionally maintained under stirring at 60° C. for one hour. Both solutions were mixed together, stirred at room temperature for 10 minutes, and then added to a perfume oil (25 g, Table 2a or Table 2b) at room temperature to form an oil phase. Oil phase was mixed with water (94.05 g), the latter comprising optionally an amino compound A (first amino-compound) or a base. Reaction mixture was stirred with an Ultra Turrax at 24,000 rpm for 30 s or 1 minute to afford an emulsion. An amino compound B (second amino-compound) (Table 3) was dissolved in water (5 g) and this solution was added dropwise to the emulsion. A solution of guanidine carbonate (30 wt % in water, 5 to 10 g) was optionally added to control pH value at about 8. The reaction mixture was stirred at 30° C. for 4 h to afford a white dispersion.

### Example 1

### Preparation of Microcapsules According to the Process of the Invention

Microcapsules were prepared according to the general protocol described previously.

### Materials

TABLE 1
List of ingredients used in the examples

| Ingredients | Function |
|---|---|
| BTC¹⁾ | Acyl chloride |
| Isopthatloyl chloride²⁾ | Acyl chloride |
| mXDA³⁾ | Amino compound |
| L-lysine⁴⁾ | Amino compound |
| EDA⁵⁾ | Amino compound |
| Cystamine Dihydrochloride⁶⁾ | Amino compound |
| Diethylene triamine⁷⁾ | Amino compound |
| Spermine dihydrate⁸⁾ | Amino compound |
| Guanidine carbonate⁹⁾ | Amino compound |
| Cystine ditertbutyl ester¹⁰⁾ | Amino compound |
| Zein¹¹⁾ | Colloidal stabilizer |
| Gum arabic¹²⁾ | Colloidal stabilizer |
| Bovin Serum Albumin¹³⁾ | Colloidal stabilizer |
| White Egg¹⁴⁾ | Colloidal stabilizer |
| Bio Pro 2E063¹⁵⁾ | Colloidal stabilizer |
| Bio Pur β-lactoglobulin¹⁶⁾ | Colloidal stabilizer |
| Sodium Caseinate¹⁷⁾ | Colloidal Stabilizer |
| Lactose¹⁸⁾ | Colloidal Stabilizer |
| Sodium bicarbonate¹⁹⁾ | Base |
| Sodium Carbonate²⁰⁾ | Base |
| Sodium Hydroxide²¹⁾ | Base |

¹⁾1,3,5-benzene tricarbonyl chloride; origin: Aldrich, Switzerland
²⁾Isopthatloyl chloride²⁾; origin: Aldrich, Switzerland
³⁾m-xylylyene diamine; origin: Aldrich, Switzerland
⁴⁾L-Lysine; origin: Aldrich, Switzerland
⁵⁾Ethylene diamine; origin: Aldrich, Switzerland
⁶⁾Cystamine Dihydrochloride; origin: Aldrich, Switzerland
⁷⁾Diethylene triamine; origin: Aldrich, Switzerland
⁸⁾Spermine dihydrate; origin: Aldrich, Switzerland
⁹⁾Guanidine carbonate; origin: Aldrich, Switzerland
¹⁰⁾Cystine ditertbutyl ester; origin: Aldrich, Switzerland
¹¹⁾Zein; origin: Aldrich, Switzerland
¹²⁾Acacia gum Superstab AA, origin: Nexira, France
¹³⁾Bovin Serum Albumin; origin: Aldrich, Switzerland
¹⁴⁾White Egg
¹⁵⁾Bio Pro 2E063; origin: Agropur Inc. USA
¹⁶⁾Bio Pur β-lactoglobulin; origin: Danisco
¹⁷⁾Sodium Caseinate; origin: Aldrich, Switzerland
¹⁸⁾Lactose; origin: Aldrich, Switzerland
¹⁹⁾Sodium bicarbonate; origin: Aldrich, Switzerland
²⁰⁾Sodium carbonate; origin: Aldrich, Switzerland
²¹⁾Sodium Hydroxide; origin: Aldrich, Switzerland

### Perfume Oil Composition:

TABLE 2a
Perfume oil A composition

| Raw Materials | % in oil |
|---|---|
| 2,4-Dimethyl-3-cyclohexene-1-carbaldehyde | 3.30% |
| Allyl Heptanoate | 5.50% |
| Allyl amyl glycolate | 10.99% |
| Delta Damascone | 1.65% |
| Verdyl acetate | 20.30% |
| Hedione ®¹⁾ | 4.95% |
| Iso E Super ®²⁾ | 16.49% |
| Ald. Hexylcinnamique | 9.89% |
| Ethyl-2-methylvalerate | 3.3% |
| Lilial | 21.98% |
| (3Z)-3-hexen-1-y1 butyrate | 1.1% |
| Ambrox ®³⁾ | 0.55% |
| Total | 100% |

¹⁾Methyl dihydrojasmonate, Firmenich SA, Geneva, Switzerland
²⁾1-(octahydro-2,3,8,8-tetramethy1-2-naphtaleny1)-1-ethanone, International Flavors & Fragrances, USA
³⁾(−)-(8R)-8,12-epoxy-13,14,15,16-tetranorlabdane, Firmenich SA, Geneva, Switzerland

TABLE 2b
Perfume oil B composition

| Ingredient | Parts |
|---|---|
| Ethyl 2-methyl-pentanoate | 3.2 |
| Eucalyptol | 7.8 |
| Aldehyde C10 | 0.75 |
| 2,4-Dimethy1-3-cyclohexene- | 0.75 |
| 1-carbaldehyde¹⁾ | |
| Citronellyl nitrile | 4.3 |
| Isobornyl acetate | 3.0 |
| Verdox²⁾ | 9.8 |
| Citronellyl acetate | 1.3 |
| 2-methylundecanal | 3.0 |
| Diphenyloxide | 0.8 |
| Aldehyde C12 | 1.3 |
| Dicyclopentadiene acetate | 9.85 |
| Ionone beta | 3.3 |
| Undecalactone gamma | 18.75 |
| Hexyl salicylate | 15.9 |
| Benzyl salicylate | 16.2 |

¹⁾Origin: Firmenich SA, Geneva, Switzerland
²⁾Trademark from IFF; 2-tert-butyl-1-cyclohexyl acetate

Capsules A: Preparation of Capsules A with Bovin Serum Albumin as Stabilizer

TABLE 3
Capsule A compositions

| | | | | | [NH₂]/ |
|---|---|---|---|---|---|
| | | Base | | Amine | [COCl] |
| Capsules | Base | (g) | Amine | (g) | (mol/mol) |
| A1 | No base | 0 | Ethylene diamine | 0.48 | 0.8 |
| A2 | No base | 0 | 3-aminopropyl | 3.76 | 0.8 |
| | | | triethoxysilane | | |
| A3 | Sodium | 0.8 | mXDA | 1.5 | 1.5 |
| | carbonate | | | | |
| A4 | Sodium | 0.8 | mXDA | 1 | 1 |
| | bicarbonate | | | | |
| A5 | Sodium | 0.5 | mXDA | 1.4 | 1.5 |
| | bicarbonate | | | | |

Capsules B: Preparation of Capsules B with Bovin Serum Albumin

TABLE 4
Capsule B compositions

| | 1^{st} amino | | 2^{nd} amino | |
|---|---|---|---|---|
| Capsules | compound | (g) | compound | (g) |
| B1 | L-Lysine | 2.5 | EDA | 0.48 |
| B2 | L-Lysine | 2.5 | Diethylene triamine | 0.82 |
| B3 | L-Lysine | 2.5 | m-XDA | 1.08 |
| B4 | L-Lysine | 2.5 | Spermine dihydrate | 1.9 |
| B5 | L-Lysine | 2.5 | Guanidine | 0.72 |
| | | | carbonate | |
| B6 | 3-aminopropyl | 3.76 | EDA | 0.48 |
| | triethoxysilane | | | |
| B7 | 3-aminopropyl | 3.76 | Diethylene triamine | 0.82 |
| | triethoxysilane | | | |
| B8 | L-Lysine | 2.5 | Cystamine | 1.8 |
| | | | dihydrochloride | |
| B9 | L-Lysine | 2.5 | L-cystine bis(tert- | 3.4 |
| | | | butyl)ester | |
| | | | dihydrochloride | |
| B10 | 3-aminopropyl | 3.76 | Cystamine | 1.8 |
| | triethoxysilane | | dihydrochloride | |
| B11** | L-Lysine | 2.5 | EDA | 0.24 |
| B12** | 3-aminopropyl | 3.76 | Spermine dihydrate | 1.9 |
| | triethoxysilane | | | |
| B13** | 3-aminopropyl | 3.76 | Ethylene diamine | 0.48 |
| | triethoxysilane | | | |

**Protein and acid chloride mixed at 60° C. for 1 hour

Capsules C: Preparation of Capsules C with Sodium Caseinate

TABLE 5
Capsule C compositions

| | 1^{st} amino | | 2^{nd} amino | | Mass Colloidal |
|---|---|---|---|---|---|
| Capsules | compound | (g) | compound | (g) | Stabilizer |
| C1 | L-Lysine | 2.5 | EDA | 0.48 | 0.95 |
| C2 | L-Lysine | 2.5 | EDA | 0.24 | 0.95 |
| C3 | L-Lysine | 2.5 | EDA | 0.24 | 2 |
| C4 | L-Lysine | 2.5 | — | — | 2 |
| C5 | L-Lysine | 2.5 | Cystamine | 0.90 | 0.95 |
| | | | Dihydrochloride | | |

Capsules D: Preparation of Capsules D with Different Stabilizers and Combination of them in the Oil Phase
Capsules D were prepared in the presence of different proteins and diamines according the protocol used to prepare capsules B in the presence of L-Lysine (2.5 g) as an amino compound A and ethylene diamine (0.48 g or 0.24 g) as an amino compound B.

TABLE 6
Capsules D compositions

| Capsules | Protein | Protein (g) |
|---|---|---|
| D1 | Gum Arabic | 0.95 |
| D2 | Zein | 0.95 |
| D3 | White Egg | 0.95 |
| D4 | New Rice | 0.95 |
| D5 | Bi Pro LE 063-18-420 | 0.95 |
| D6 | Bio Pure β-lacto globulin | 0.95 |
| D7 | Sodium Caseinate/Bi Pro | 2 |
| | LE 063-18-420 | |
| D8 | Sodium Caseinate/ | 2 |
| | Lactose | |

Capsules E: Preparation of Capsules E with a Mixture of Acyl Chloride and Caseinate in the Oil Phase
Capsules E were prepared in the presence of Caseinate and diamines according the protocol used to prepare capsules B in the presence of L-Lysine (2.5 g) as an amino compound A and ethylene diamine (0.48 g or 0.24 g) as an amino compound B.

TABLE 7
Capsules E compositions

| | | Molarity |
|---|---|---|
| Capsules | Acyl chloride | OCl mmol |
| E1 | Isopthaloyl chloride/BTC | 10/10 |
| E8 | Isopthaloyl chloride/BTC | 5/15 |

Capsules F: Preparation of Capsules F with Only One Amino Compound and Caseinate and Mixture of Proteins in the Oil Phase
Capsules F were prepared in the presence of protein of mixture of proteins according the protocol used to prepare capsules B with only the presence of L-Lysine (2.5 g) as an amino compound A in the water phase during the process of emulsification. No additional amino compound B is added after the emulsion.

TABLE 8
Capsules F compositions

| Capsules | Protein | (g) |
|---|---|---|
| F1 | Caseinate | 2 |
| F2 | 75% Caseinate/25% | 2 |
| | Whey protein | |

### Example 2

### Storage Stability in a Fabric Softener Composition

The storage stability of the capsules in fabric softener was evaluated. Capsule dispersion (0.27 g) of the present invention (with encapsulated perfume oil B) was diluted in the fabric softener composition described in Table 9 (29.73 g). The softener was stored for up to one month at 37° C. The amount of perfume having leaked out of the capsules was then measured by solvent extraction and GC-FID analysis (Table 10).

TABLE 9
Fabric Softener composition

| Product | Wt % |
|---|---|
| Stepantex VL 90A | 8.88 |
| Calcium Chloride Sol. 10% | 0.36 |
| Proxel GXL | 0.04 |
| Perfume | 1 |
| Water | 89.72 |
| TOTAL | 100 |

TABLE 10
Oil leakage of microcapsules in
Fabric Softener composition

| | Leakage 3 | Leakage 30 |
|---|---|---|
| Capsules | days (%) | days (%) |
| A1 | 7 | 25 |
| A3 | 14 | 25 |
| A4 | 4 | 5 |
| A5 | 8 | 8 |
| A2 | 9.5 | 20.8 |
| B8 | 10.8 | 16.6 |
| B11 | 8.2 | 19.7 |
| C1 | 22 | 32 |
| C2 | 11 | 25 |
| C3 | 13 | 23 |
| C4 | 25 | 49 |
| C5 | 24 | 33 |
| D1 | 21 | 29 |
| D4 | 22 | 37 |
| D5 | 19 | 38 |
| D6 | 9 | 22 |
| D7 | 11 | 22 |
| D8 | 16 | 27 |
| E1 | 21 | 50 |
| E2 | 20 | 48 |
| F1 | 25 | 49 |
| F2 | 20 | 56 |

One can conclude that microcapsules of the invention show a satisfactory stability in a challenging medium.

### Example 3

### Spray-Dried Microcapsules Preparation

Emulsions A-E having the following ingredients are prepared.

TABLE 11
Composition of Emulsions A-E and composition of granulated powder
A-E after spray-drying

| Ingredients | | | | | |
|---|---|---|---|---|---|
| | Emulsion | Emulsion | Emulsion | Emulsion | Emulsion |
| | A | B | C | D | E |
| Modified | 2.6% | 2.6% | 2.6% | 12.5% | 2% |
| starch ¹⁾ | | | | | |
| Maltodextrin ²⁾ | 26.8% | 22.8% | 19.3% | 0% | 19.1% |
| Maltose ³⁾ | 0% | 0% | 0% | 7.9% | 0% |
| Citric Acid | 0% | 0% | 0% | 1% | 0% |
| Tripotassium | 0% | 0% | 0% | 1.9% | 0% |
| Citrate | | | | | |
| Microcapsules | 12.0% | 24% | 37.0% | 8.9% | 56.2% |
| A-F | | | | | |
| Silica ⁴⁾ | 1.1% | 1.1% | 1.1% | 0% | 0% |
| Free | 0% | 0% | 0% | 11% | 0% |
| Perfume C ⁵⁾ | | | | | |
| Water | 57.6% | 49.6% | 40.1% | 56.9% | 22.7% |
| | Granule | Granule | Granule | Granule | Granule |
| | A | B | C | D | E |
| Modified | 7.5% | 7.4% | 7.2% | 31.6% | 4.9% |
| starch ¹⁾ | | | | | |
| Maltodextrin ²⁾ | 77.4% | 65.5% | 53.8% | 0% | 44.7% |
| Maltose | 0% | 0% | 0% | 20.9% | |
| Citric Acid | 0% | 0% | 0% | 2.6% | 0% |
| Tripotassium | 0% | 0% | 0% | 4.9% | 0% |
| citrate | | | | | |
| Encapsulated | 0% | 0% | 0% | 28.1% | 0% |
| perfume C | | | | | |
| Microcapsules | 12.% | 24.1% | 36.1% | 9.8% | 48.4% |
| I-L | | | | | |
| Silica | 3.0 | 3.0% | 2.9% | 2.0% | 2% |
| Fragrance | 10.1% | 20.1% | 30% | 35.8% | 40.2% |
| loading in | | | | | |
| powder after | | | | | |
| spray-drying | | | | | |

¹⁾ CapsulTM, Ingredion
²⁾ Maltodextrin 10DE origin: Roquette
³⁾ Maltose, Lehmann & Voss
⁴⁾ Silica, Evonik
⁵⁾ see table 12

TABLE 12
Composition of Perfume C

| Component | % |
|---|---|
| ACÉTATE DE 4-(1,1-DIMÉTHYLÉTHYL)-1- | 14.50 |
| CYCLOHEXYLE ¹⁾ | |
| LINALOL BJ | 10.50 |
| LILIAL ®²⁾ | 10.00 |
| ISO E SUPER ³⁾ | 10.00 |
| CITRONELLYL NITRILE | 9.00 |
| DIPHENYLOXYDE | 6.50 |
| ISOBORNYL ACETATE | 6.00 |
| BETA IONONE | 6.00 |
| TRICYCLO[5.2.1.0~2,6~]DEC-3-EN-8-YL ACETATE (A) + | 5.50 |
| TRICYCLO[5.2.1.0~2,6~]DEC-4-EN-8-YL ACETATE (B) ⁴⁾ | |
| ETHER MT | 4.00 |
| HEDIONE ® ⁵⁾ | 4.00 |
| GERANIOL 60 | 3.00 |
| CITRAL | 2.50 |
| ALDEHYDE C 10 | 2.50 |
| ALLYL HEPTANOATE | 2.50 |
| ETHYL METHYL-2-BUTYRATE | 1.50 |
| GERANYL ACETATE | 1.00 |
| 2,4-DIMETHYL-3-CYCLOHEXENE-1-CARBALDEHYDE ⁶⁾ | 1.00 |

¹⁾ Firmenich SA, Switzerland
²⁾3-(4-tert-butylphenyl)-2-methylpropanal, Givaudan SA, Vernier, Switzerland
³⁾ 1-(octahydro-2,3,8,8-tetramethyl-2-naphtalenyl)-1-ethanone, International Flavors & Fragrances, USA
⁴⁾ Firmenich SA, Switzerland
⁵⁾ Methyl dihydrojasmonate, Firmenich SA, Switzerland
⁶⁾ Firmenich SA, Switzerland

Components for the polymeric matrix (Maltodextrin and Capsul™, or Capsul™, citric acid and tripotassium citrate) are added in water at 45-50° C. until complete dissolution.
For emulsion D, free perfume C is added to the aqueous phase.
Microcapsules slurry is added to the obtained mixture. Then, the resulting mixture is then mixed gently at 25° C. (room temperature).
Granulated powder A-E are prepared by spray-drying Emulsion A-E using a Sodeva Spray Dryer (Origin France), with an air inlet temperature set to 215° C. and a throughput set to 500 ml per hour. The air outlet temperature is of 105° C. The emulsion before atomization is at ambient temperature.

### Example 4

### Liquid Scent Booster Composition

A sufficient amount of microcapsule slurry A-F is weighed and mixed in a liquid scent booster (Table 13) to add the equivalent of 0.2% perfume.

TABLE 13
Liquid scent booster composition

| | Amount (% wt) | | | | | |
|---|---|---|---|---|---|---|
| Ingredients | 1 | 2 | 3 | 4 | 5 | 6 |
| Water | 71.20% | 89.5% | 78.8% | 79.4% | 70% | 70% |
| Propylene glycol | 20.30% | — | — | — | 20% | 20% |
| Polyethylene | 4.00% | 6% | | | | |
| glycol ethers of | | | | | | |
| decyl alcohol¹⁾ | | | | | | |
| Polyethylene | | | | | 4.00% | 4.00% |
| glycol ether of | | | | | | |
| Lauryl Alcohol²⁾ | | | | | | |
| alkyl poly- | | | 8.30% | 7.7% | | |
| glucoside | | | | | | |
| C8-C10³⁾ | | | | | | |
| Deceth-3¹⁾ | 1.50% | | | | | |
| Lauryl lactate | | | | 1% | | |
| Lauric acid | | 1.5% | 1.60% | | | |
| Glyceryl | | | | | 3.00% | 3.00% |
| Caprylate | | | | | | |
| Fragrance | 3.00% | 3.0% | 3.00% | 3.00% | 3.00% | 0% |

¹⁾Deceth-8; trademark and origin: KLK Oleo
²⁾Laureth-9;; trademark and origin
³⁾Plantacare 2000UP; trademark and origin: BASF

Different ringing gel compositions are prepared (compositions 1-6) according to the following protocol.

In a first step, the aqueous phase (water), the solvent (propylene glycol) if present and surfactants are mixed together at room temperature under agitation with magnetic stirrer at 300 rpm for 5 min.

In a second step, the linker is dissolved in the hydrophobic active ingredient (fragrance) at room temperature under agitation with magnetic stirrer at 300 rpm. The resulting mixture is mixed for 5 min.

Then, the aqueous phase and the oil phase are mixed together at room temperature for 5 min leading to the formation of a transparent or opalescent ringing gel.

### Example 5

### Liquid Detergent Composition

A sufficient amount of microcapsule slurry A-F is weighed and mixed in a liquid detergent (Table 14) to add the equivalent of 0.2% perfume.

TABLE 14
Liquid detergent composition

| | Concentration |
|---|---|
| Ingredients | [wt %] |
| Sodium C₁₄₋₁₇ Alkyl Sec | 7 |
| Sulfonate¹⁾ | |
| Fatty acids, C₁₂₋₁₈ and C₁₈- | 7.5 |
| unsaturated²⁾ | |
| C_{12/14} fatty alcohol polyglycol | 17 |
| ether with 7 mol EO³⁾ | |
| Triethanolamine | 7.5 |
| Propylene Glycol | 11 |
| Citric acid | 6.5 |
| Potassium Hydroxyde | 9.5 |
| Properase L⁴⁾ | 0.2 |
| Puradax EG L⁴⁾ | 0.2 |
| Purastar ST L⁴⁾ | 0.2 |
| Acrylates/Steareth-20 | 6 |
| Methacrylate | |
| structuring Crosspolymer⁵⁾ | |
| Deionized Water | 27.4 |

¹⁾Hostapur SAS 60; Origin: Clariant
²⁾Edenor K 12-18; Origin: Cognis
³⁾Genapol LA 070; Origin: Clariant
⁴⁾Origin: Genencor International
⁵⁾Aculyn 88; Origin: Dow Chemical

### Example 6

### Powder Detergent Composition

A sufficient amount of granules A-E is weighed and mixed in a powder detergent composition (Table 15) to add the equivalent of 0.2% perfume.

TABLE 15
Powder detergent composition

| Ingredients | Part |
|---|---|
| Anionic (Linear Alkyl Benzene | 20% |
| Sulphonates) | |
| Nonionics (Alcohol Ethoxylates (5-9 | 6% |
| ethylene oxide) | |
| Builders (zeolites, sodium carbonate) | 25% |
| Silicates | 6% |
| Sodium Sulphate | 35% |
| Others (Enzymes, Polymers, Bleach) | 7.5% |
| Spray-dried granule powder A-E | 0.5% |

### Example 7

### Concentrated all Purpose Cleaner Composition

A sufficient amount of microcapsule slurry A-F is weighed and mixed in a concentrated all-purpose cleaner composition (Table 16) to add the equivalent of 0.2% perfume.

TABLE 16
concentrated all-purpose cleaner composition

| Ingredients | Amount (% wt) | Function |
|---|---|---|
| Ethoxylated Alcohol (C9- | 20 | Non-ionic |
| C11, 8EO) ⁽¹⁾ | | surfactant |
| Sodium Dodecyl Benzene | 16 | Anionic surfactant |
| Sulfonate ⁽²⁾ | | |
| Sodium Cumene | 8 | Hydrotrope |
| Sulfonate ⁽³⁾ | | |
| Methyl chloro | 0.8% | preservative |
| isothiazolinone Methyl | | |
| isothiazolinone 3.3:1 ⁽⁴⁾ | | |
| Water | 55.9 | solvent |

⁽¹⁾ Neodol 91-8 ®; trademark and origin: Shell Chemical
⁽²⁾ Biosoft D-40 ®; trademark and origin: Stepan Company
⁽³⁾ Stepanate SCS ®; trademark and origin: Stepan Company
⁽⁴⁾ Kathon CG ®; trademark and origin : Dow Chemical Company

All ingredients are mixed together and then the mixture was diluted with water to 100%.

### Example 8

### Solid Scent Booster Composition

The following compositions are prepared.

TABLE 17
Salt-based solid scent booster compositions

| Ingredients | Part |
|---|---|
| Sodium chloride | 95 |
| Spray-dried granule powder A-E | 5 |

TABLE 18
Urea-based solid scent booster compositions

| Ingredients | Part |
|---|---|
| Urea (beads) | 86 |
| Spray-dried granule powder A-E | 8 |
| Bentonite | 3 |
| Perfume | 3 |

### Example 9

### Shampoo Composition

A sufficient amount of microcapsule slurry A-F is weighed and mixed in a shampoo composition (Table 19) to add the equivalent of 0.2% perfume.

TABLE 19
Shampoo composition

| | | Concentration |
|---|---|---|
| | Ingredients | [wt %] |
| A | Water deionized | 44.4 |
| | Polyquaternium-10 ¹⁾ | 0.3 |
| | Glycerin 85% ²⁾ | 1 |
| | DMDM Hydantoin ³⁾ | 0.2 |
| B | Sodium Laureth Sulfate ⁴⁾ | 28 |
| | Cocamidopropyl Betaine ⁵⁾ | 3.2 |
| | Disodium Cocoamphodiacetate ⁶⁾ | 4 |
| | Ethoxy (20) Stearyl Alcohol ⁶⁾ | 1 |
| C | Sodium Laureth Sulfate ⁴⁾ | 3 |
| | Glyceryl Laureate ⁷⁾ | 0.2 |
| D | Water deionized | 1 |
| | Sodium Methylparaben ⁸⁾ | 0.1 |
| E | Sodium Chloride 10% aqueous sol. | 15 |
| | Citric acid 10% aqueous sol. till pH 5.5-6 | q.s. |
| | Perfume | 0.5 |
| | TOTAL: | 100 |

¹⁾ Ucare Polymer JR-400, Noveon
2) Schweizerhall
³⁾ Glydant, Lonza
⁴⁾ Texapon NSO IS, Cognis
⁵⁾ Tego Betain F 50, Evonik
⁶⁾ Amphotensid GB 2009, Zschimmer & Schwarz
⁷⁾ Monomuls 90 L-12, Gruenau
⁸⁾ Nipagin Monosodium, NIPA

Polyquaternium-10 is dispersed in water. The remaining ingredients of phase A are mixed separately by addition of one after the other while mixing well after each adjunction. Then this pre-mix is added to the Polyquaternium-10 dispersion and was mixed for 5 min. Then Phase B and the premixed Phase C (heat to melt Monomuls 90L-12 in Texapon NSO IS) are added. The mixture is mixed well. Then, Phase D and Phase E are added while agitating. The pH was adjusted with citric acid solution till pH: 5.5-6.0.

### Example 10

### Shampoo Composition

A sufficient amount of microcapsule slurry A-F is weighed and mixed in a shampoo composition (Table 20) to add the equivalent of 0.200 perfume.

TABLE 20
Shampoo composition

| | | Concentration |
|---|---|---|
| | Ingredients | [wt %] |
| A | Water deionized | 45.97 |
| | Tetrasodium EDTA ¹⁾ | 0.05 |
| | Guar Hydroxypropyltrimonium Chloride ²⁾ | 0.05 |
| | Polyquaternium-10 ³⁾ | 0.075 |
| B | NaOH 10% aqueous sol. | 0.3 |
| C | Ammonium Lauryl Sulfate ⁴⁾ | 34 |
| | Ammonium Laureth Sulfate ⁵⁾ | 9.25 |
| | Cocamidopropyl Betaine ⁶⁾ | 2 |
| | Dimethicone (&) C12-13 Pareth-4 (&) C12-13 | 2.5 |
| | Pareth-23 (&) Salicylic Acid ⁷⁾ | |
| D | Cetyl Alcohol ⁸⁾ | 1.2 |
| | Cocamide MEA ⁹⁾ | 1.5 |
| | Glycol Distearate ¹⁰⁾ | 2 |
| E | Methylchloroisothiazolinone & | 0.1 |
| | Methylisothiazolinone ¹¹⁾ | |
| | D-Panthenol 75% ¹²⁾ | 0.1 |
| | Water deionized | 0.3 |
| F | Sodium Chloride 25% aqueous sol. | 0.6 |
| | TOTAL: | 100 |

¹⁾ EDETA B Powder, BASF
²⁾ Jaguar C14 S, Rhodia
³⁾ Ucare Polymer JR-400, Noveon
⁴⁾ Sulfetal LA B-E, Zschimmer & Schwarz
⁵⁾ Zetesol LA, Zschimmer & Schwarz
⁶⁾ Tego Betain F 50, Evonik
⁷⁾ Xiameter MEM-1691, Dow Corning
⁸⁾ Lanette 16, BASF
⁹⁾ Comperlan 100, Cognis
¹⁰⁾ Cutina AGS, Cognis
¹¹⁾ Kathon CG, Rohm & Haas
¹²⁾ D-Panthenol, Roche

A premix comprising Guar Hydroxypropyltrimonium Chloride and Polyquaternium-10 are added to water and Tetrasodium EDTA while mixing. When the mixture is homogeneous, NaOH is added. Then, Phase C ingredients are added and the mixture was heat to 75° C. Phase D ingredients are added and mixed till homogeneous. The heating is stopped and temperature of the mixture is decreased to RT. At 45° C., ingredients of Phase E while mixing final viscosity is adjusted with 25% NaCl solution and pH of 5.5-6 is adjusted with 10% NaOH solution.

### Example 11

### Rinse-Off Hair Composition

A sufficient amount of microcapsule slurry A-F is weighed and mixed in a rinse-off composition (Table 21) to add the equivalent of 0.2% perfume.

TABLE 21
rinse-off composition

| | | Concentration |
|---|---|---|
| | Ingredients | [wt %] |
| A | Water deionized | 81.8 |
| | Behentrimonium Chloride ¹⁾ | 2.5 |
| | Hydroxyethylcellulose ²⁾ | 1.5 |
| B | Cetearyl Alcohol ³⁾ | 4 |
| | Glyceryl Stearate (and) PEG-100 | 2 |
| | Stearate ⁴⁾ | |
| | Behentrimonium Methosulfate (and) Cetyl | |
| | alcohol (and) Butylene Glycol ⁵⁾ | 4 |
| | Ethoxy (20) Stearyl Alcohol ⁶⁾ | 1 |
| C | Amodimethicone (and) Trideceth-12 (and) | 3 |
| | Cetrimonium Chloride ⁷⁾ | |
| | Chlorhexidine Digluconate ⁸⁾ 20% | 0.2 |
| | aqueous solution | |
| D | Citric acid 10% aqueous sol. till pH 3.5-4 | q.s. |
| | TOTAL: | 100 |

¹⁾ Genamin KDMP, Clariant
²⁾ Tylose H10 Y G4, Shin Etsu
³⁾ Lanette O, BASF
⁴⁾ Arlacel 165, Croda
⁵⁾ Incroquat Behenyl TMS-50-PA- (MH), Croda
⁶⁾ Brij S20, Croda
⁷⁾ Xiameter MEM-949, Dow Corning
⁸⁾ Alfa Aesar

Ingredients of Phase A are mixed until an uniform mixture was obtained. Tylose is allowed to completely dissolve. Then the mixture is heated up to 70-75° C. Ingredients of Phase B are combined and melted at 70-75° C. Then ingredients of Phase B are added to Phase A with good agitation and the mixing is continued until cooled down to 60° C. Then, ingredients of Phase C are added while agitating and keeping mixing until the mixture cooled down to 40° C. The pH is adjusted with citric acid solution till pH: 3.5-4.0.

### Example 12

### Antiperspirant Spray Anhydrous Composition

A sufficient amount of microcapsule slurry A-F is weighed and mixed in an antiperspirant spray anhydrous composition (Table 22) to add the equivalent of 0.2% perfume.

TABLE 22
antiperspirant spray anhydrous composition

| Ingredient | Amount (wt %) |
|---|---|
| Cyclomethicone¹⁾ | 53.51 |
| Isopropyl miristate | 9.04 |
| Silica²⁾ | 1.03 |
| Quaternium-18-Hectorite³⁾ | 3.36 |
| Aluminium Chlorohydrate⁴⁾ | 33.06 |

¹⁾Dow Corning ® 345 Fluid; trademark and origin: Dow Corning
²⁾Aerosil ® 200 ; trademark and origin : Evonik
³⁾Bentone ® 38; trademark and origin : Elementis Specialities
⁴⁾Micro Dry Ultrafine; origin : Reheis

Using a high speed stirrer, Silica and Quaternium-18-Hectorite are added to the Isopropyl miristate and Cyclomethicone mixture. Once completely swollen, Aluminium Chlorohydrate is added portion wise under stirring until the mixture was homogeneous and without lumps. The aerosol cans are filled with 25% Suspension of the suspension and 75% of Propane/Butane (2.5 bar).

### Example 13

### Antiperspirant Spray Emulsion Composition

A sufficient amount of microcapsule slurry A-F is weighed and mixed in antiperspirant spray emulsion composition (Table 23) to add the equivalent of 0.2% perfume.

TABLE 23
antiperspirant spray emulsion composition

| Ingredient | Amount (wt %) |
|---|---|
| Polysorbate 65¹⁾ (Part A) | 0.95 |
| Polyglyceryl-2 | 1.05 |
| dipolyhydroxystearate²⁾ (Part A) | |
| Cetyl PEG/PPG-10/1 | 2.75 |
| Dimethicone³⁾ (Part A) | |
| Cyclomethicone⁴⁾ (Part A) | 16.4 |
| Isopropylisostearate⁵⁾ (Part A) | 4.5 |
| Phenoxyethanol⁶⁾ (Part A) | 0.5 |
| Ethylhexylglycerin⁷⁾ (Part A) | 0.2 |
| C12-15 Alkyl Benzoate⁸⁾ (Part A) | 5.65 |
| Silica Silylate⁹⁾ (Part A) | 0.1 |
| Sodium Methylparaben¹⁰⁾ (Part B) | 0.1 |
| Aluminium Chlorohydrate¹¹⁾ (Part B) | 20 |
| Water (Part B) | 44.47 |
| Fragrance (Part C) | 3.33 |

¹⁾Tween 65; trademark and origin : CRODA
²⁾Dehymuls PGPH; trademark and origin : BASF
³⁾Abil EM-90; trademark and origin : BASF
⁴⁾Dow Corning 345 fluid; trademark and origin : Dow Corning
⁵⁾Crodamol ipis; trademark and origin : CRODA
⁶⁾Phenoxyethanol; trademark and origin : LANXESS
⁷⁾Sensiva sc 50; trademark and origin : KRAFT
⁸⁾Tegosoft TN; trademark and origin : Evonik
⁹⁾Aerosil R 812; trademark and origin : Evonik
¹⁰⁾Nipagin mna; trademark and origin : CLARIANT
¹¹⁾Locron L; trademark and origin : CLARIANT

The ingredients of Part A and Part B are weighted separately. Ingredients of Part A are heated up to 60° C. and ingredients of Part B are heated to 55° C. Ingredients of Part B are poured small parts while continuous stirring into A. Mixture were stirred well until the room temperature was reached. Then, ingredients of part C are added. The emulsion is mixed and is introduced into the aerosol cans. The propellant is crimped and added.
Aerosol filling: 30% Emulsion: 70% Propane/Butane 2.5 bar

### Example 14

### Deodorant Spray Composition

A sufficient amount of microcapsule slurry A-F is weighed and mixed in antiperspirant deodorant spray composition (Table 24) to add the equivalent of 0.2% perfume.

TABLE 24
deodorant spray composition

| Ingredient | Amount (wt %) |
|---|---|
| Ethanol 95 % | 90.65 |
| Triclosan¹⁾ | 0.26 |
| Isopropyl miristate | 9.09 |

¹⁾Irgasan ® DP 300; trademark and origin : BASF

All the ingredients according to the sequence of the Table 24 are mixed and dissolved. Then the aerosol cans are filled, crimp and the propellant is added (Aerosol filling: 40% active solution 60% Propane/Butane 2.5 bar).

### Example 15

### Antiperspirant Roll-on Emulsion Composition

A sufficient amount of microcapsule slurry A-F is weighed and mixed in antiperspirant roll-on emulsion composition (Table 25) to add the equivalent of 0.2% perfume.

TABLE 25
antiperspirant roll-on emulsion composition

| Ingredient | Amount (wt %) |
|---|---|
| Steareth-2¹⁾ (Part A) | 3.25 |
| Steareth-21²⁾ (Part A) | 0.75 |
| PPG-15 Stearyl Ether³⁾ (Part A) | 4 |
| WATER deionised (Part B) | 51 |
| Aluminum Chlorohydrate 50% | 40 |
| aqueous solution⁴⁾ (Part C) | |
| Fragrance (Part D) | 1 |

¹⁾BRIJ 72; origin : ICI
²⁾BRIJ 721; origin : ICI
³⁾ARLAMOL E; origin : UNIQEMA-CRODA
⁴⁾LOCRON L; origin: CLARIAN

Part A and B are heated separately to 75° C.; Part A is added to part B under stirring and the mixture is homogenized for 10 minutes. Then, the mixture is cooled down under stirring; and part C is slowly added when the mixture reached 45° C. and part D when the mixture reached at 35° C. while stirring. Then the mixture is cooled down to RT.

### Example 16

### Antiperspirant Roll-on Composition

A sufficient amount of microcapsule slurry A-F is weighed and mixed in antiperspirant roll-on composition (Table 26) to add the equivalent of 0.2% perfume.

TABLE 26
antiperspirant roll-on composition

| Ingredient | QUANTITY |
|---|---|
| Water (Part A) | 45 |
| Aluminum Chlorohydrate 50% | 20 |
| aqueous solution¹⁾ (Part B) | |
| Alcohol Denat. (Ethanol | 30 |
| 96%) (Part B) | |
| Ceteareth-12²⁾ (Part C) | 2 |
| Ceteareth-30³⁾ (Part C) | 2 |
| Fragrance (Part D) | 1 |

¹⁾LOCRON L; origin: CLARIANT
²⁾EUMULGIN B-1; origin : BASF
³⁾EUMULGIN B-3; origin : BASF

The ingredients of part B are mixed in the vessel then ingredient of part A is added. Then dissolved part C in part A and B. With perfume, 1 part of Cremophor RH40 for 1 part of perfume is added while mixing well

### Example 17

### Antiperspirant Roll-on Composition

A sufficient amount of microcapsule slurry A-F is weighed and mixed in antiperspirant roll-on emulsion composition (Table 27) to add the equivalent of 0.2% perfume.

TABLE 27
antiperspirant roll-on emulsion composition

| Ingredient | Amount (wt %) |
|---|---|
| Water (Part A) | 50.51 |
| Hydroxyethylcellulose¹⁾ (Part A) | 0.71 |
| Ethanol 95 % (Part B) | 40.40 |
| 1,2-Propylene Glycol (Part B) | 5.05 |
| Triclosan²⁾ (Part B) | 0.30 |
| PEG-40 Hydrogenated | 3.03 |
| castor oil³⁾ (Part C) | |

¹⁾Natrosol ® 250 H; trademark and origin: Ashland
²⁾Irgasan ® DP 300; trademark and origin : BASF
³⁾Cremophor ® RH 40; trademark and origin : BASF

Part A is prepared by sprinkling little by little the Hydroxyethylcellulose in the water whilst rapidly stirring with the turbine. Stirring is continued until the Hydroxyethylcellulose is entirely swollen and giving a limpid gel. Then, Part B is poured little by little in Part A whilst continuing stirring until the whole is homogeneous. Part C is added.

### Example 18

### Deodorant Pump without Alcohol Formulation

A sufficient amount of microcapsule slurry A-F is weighed and mixed in the following composition (Table 28) to add the equivalent of 0.2% perfume.

TABLE 28
deodorant composition

| Ingredients | Amount (wt %) |
|---|---|
| C12-15 Alkyl | 5 |
| Lactate¹⁾ | |
| Dimethicone²⁾ | 91.6 |
| Cetyl Lactate³⁾ | 1 |
| Octyldodecanol⁴⁾ | 0.8 |
| Triclosan⁵⁾ | 0.1 |
| PERFUME | 1.5 |

¹⁾Ceraphyl 41; trademark and origin ASHLAND
²⁾DOW CORNING 200 FLUID 0.65cs; trademark and origin DOW CORNING CORPORATION
³⁾Ceraphyl 28; trademark and origin ASHLAND
⁴⁾Eutanol G; trademark and origin BASF
⁵⁾Irgasan ® DP 300; trademark and origin : BASF

All the ingredients of Table 28 are mixed according to the sequence of the table and the mixture is heated slightly to dissolve the Cetyl Lactate.

### Example 19

### Deodorant Pump with Alcohol Formulation

A sufficient amount of microcapsule slurry A-F is weighed and mixed in the following composition (Table 29) to add the equivalent of 0.2% perfume.

TABLE 29
deodorant composition

| Ingredients | Amount (wt %) |
|---|---|
| Ethyl Alcohol (Part A) | 60 |
| PEG-6 Caprylic/Capric | 2 |
| Glycerides¹⁾ (Part A) | |
| Water (Part A) | 35.6 |
| PEG-40 Hydrogenated | 0.4 |
| Castor Oil²⁾ (Part B) | |
| PERFUME (Part B) | 2 |

¹⁾Softigen 767; trademark and origin CRODA
²⁾Cremophor ® RH 40; trademark and origin : BASF

Ingredients from Part B are mixed together. Ingredients of Part A are dissolved according to the sequence of the Table and are poured into part B.

### Example 20

### Talc Formulation

A sufficient amount of granules A-F is weighed and mixed in introduced in a standard talc base: 100% talc, very slight characteristic odor, white powder, origin: LUZENAC to add the equivalent of 0.2% perfume.

### Example 21

### Shower-Gel Reference

A sufficient amount of microcapsule slurry A-F is weighed and mixed in the following composition (Table 30) to add the equivalent of 0.2% perfume.

TABLE 30
shower gel composition

| Ingredients | Amount (% wt) | Function |
|---|---|---|
| WATER deionised | 49.350 | Solvent |
| Tetrasodium EDTA ¹⁾ | 0.050 | Chelating agent |
| Acrylates Copolymer²⁾ | 6.000 | Thickener |
| Sodium C12-C15 | 35.000 | Surfactant |
| Pareth Sulfate ³⁾ | | |
| Sodium Hydroxide | 1.000 | pH adjuster |
| 20% aqueous solution | | |
| Cocamidopropyl Betaine⁴⁾ | 8.000 | Surfactant |
| Methylchloroisothiazolinone | 0.100 | Preservative |
| and Methylisothiazolinone⁵⁾ | | |
| Citric Acid (40%) | 0.500 | pH adjuster |

¹⁾ EDETA B POWDER; trademark and origin: BASF
²⁾CARBOPOL AQUA SF-1 POLYMER; trademark and origin: NOVEON
³⁾ ZETESOL AO 328 U; trademark and origin: ZSCHIMMER & SCHWARZ
⁴⁾TEGO-BETAIN F 50; trademark and origin: GOLDSCHMIDT
⁵⁾KATHON CG; trademark and origin: ROHM & HASS

Ingredients are mixed, pH is adjusted to 6-6.3 (Viscosity: 4500 cPo+/−1500 cPo (Brookfield RV/Spindle #4/20 RPM)).

### Example 22

### Shower-Gel Composition

A sufficient amount of microcapsule slurry A-F is weighed and mixed in the following composition (Table 31) to add the equivalent of 0.2% perfume.

TABLE 31
shower gel composition

| Ingredients | Amount (% wt) | Function |
|---|---|---|
| WATER deionized | 52.40 | Solvent |
| Tetrasodium EDTA ¹⁾ | 0.10 | Chelating agent |
| Sodium Benzoate | 0.50 | Preservative |
| Propylene Glycol | 2.00 | Solvent |
| Sodium C12-C15 | 35.00 | Surfactant |
| Pareth Sulfate ²⁾ | | |
| Cocamidopropyl Betaine³⁾ | 8.00 | Surfactant |
| Polyquaternium-7⁴⁾ | 0.20 | Conditioning agent |
| Citric Acid (40%) | 1.00 | pH adjuster |
| Sodium Chloride | 0.80 | Viscosity adjuster |

¹⁾ EDETA B POWDER; trademark and origin: BASF
²⁾ ZETESOL AO 328 U; trademark and origin: ZSCHIMMER & SCHWARZ
³⁾TEGO-BETAIN F 50; trademark and origin: GOLDSCHMIDT
⁴⁾MERQUAT 550; trademark and origin: LUBRIZOL

Ingredients are mixed, pH is adjusted to 4.5 (Viscosity: 3000 cPo+/−1500 cPo (Brookfield RV/Spindle #4/20 RPM)).

### Example 23

### Shower-Gel Composition

A sufficient amount of microcapsule slurry A-F is weighed and mixed in the following composition (Table 32) to add the equivalent of 0.2% perfume.

TABLE 32
shower gel composition

| Ingredients | Amount (% wt) | Function |
|---|---|---|
| WATER deionized | 50.950 | Solvent |
| Tetrasodium EDTA ¹⁾ | 0.050 | Chelating agent |
| Sodium Benzoate | 0.500 | Preservative |
| Glycerin 86% | 3.500 | Solvent |
| Sodium Laureth Sulfate ²⁾ | 27.000 | Surfactant |
| Polyquaternium-7³⁾ | 1.000 | Conditioning Agent |
| Coco-Betaine⁴⁾ | 6.000 | Surfactant |
| PEG-120 Methyl | 1.000 | Thickener |
| Glucose trioleate⁵⁾ | | |
| Citric Acid (40%) | 1.000 | pH adjuster |
| Glycol Distearate & Laureth-4 | 3.000 | Pearlizing agent |
| & Cocamidopropyl Betaine⁶⁾ | | |
| Sodium Chloride 20% | 5.000 | Viscosity adjuster |
| PEG-40 Hydrogenated | 1.000 | Viscosity adjuster |
| Castor Oil⁷⁾ | | |

¹⁾ EDETA B POWDER; trademark and origin: BASF
²⁾ Texapon NSO IS; trademark and origin: COGNIS
³⁾MERQUAT 550; trademark and origin: LUBRIZOL
⁴⁾DEHYTON AB-30; trademark and origin: COGNIS
⁵⁾GLUCAMATE LT; trademark and origin: LUBRIZOL
⁶⁾EUPERLAN PK 3000 AM; trademark and origin: COGNIS
⁷⁾CREMOPHOR RH 40; trademark and origin: BASF

Ingredients are mixed, pH is adjusted to 4.5 (Viscosity: 4000 cPo+/−1500 cPo (Brookfield RV/Spindle #4/20 RPM))

### Example 24

### Hand Dishwash

A sufficient amount of microcapsule slurry A-F is weighed and mixed in the following composition (Table 33) to add the equivalent of 0.2% perfume.

TABLE 33
Hand dishwash composition

| Ingredients | Amount (% wt) | Function |
|---|---|---|
| Linear alkylbenzene | 20 | Anionic |
| sulfonic acid ⁽¹⁾ | | surfactant |
| Diethanolamide ⁽²⁾ | 3.5 | Foam booster |
| Sodium | 3.4 | pH Adjuster/ |
| Hydroxide (50%) ⁽³⁾ | | neutralizer |
| Secondary alcohol | 2.5 | Non-ionic |
| ethoxolate ⁽⁴⁾ | | surfactant |
| Sodium xylene sulfonate | 6.3 | Hydrotrope |
| Water | 64.3 | Solvent |

⁽¹⁾ Biosoft S-118 ®; trademark and origin: Stepan Company
⁽²⁾ Ninol 40-CO ®; trademark and origin: Stepan Company
⁽³⁾ Stepanate SXS ®; trademark and origin: Stepan Company
⁽⁴⁾ Tergitol 15-S-9 ®; trademark and origin : Dow Chemical Company

Water with sodium hydroxide and diethanolamide are mixed. LAS is added. After the LAS is neutralized, the remaining ingredients are added. The pH was Checked (=7-8) and adjusted if necessary.

### Example 25

### Toothpaste Formulation

A sufficient amount of microcapsule slurry M (corresponding to microcapsules A except that a menthol flavor is encapsulated) is weighed and mixed in the following composition (Table 34) to add the equivalent of 0.2% flavor.

TABLE 34
Toothpaste formulation

| Ingredients | Amount (% wt) |
|---|---|
| Polyethylene glycol 400 | 2.0% |
| Xanthan Gum | 0.6% |
| Sorbitol 70% Solution | 50% |
| Sodium Fluoride | 0.220% |
| Sodium Benzoate | 0.2% |
| Water | 15.230% |
| Hydrated Silica¹⁾ | 22.0% |
| Hydrated Silica²⁾ | 7.0% |
| Titanium Dioxide CI77891 | 0.5% |
| Sodium Lauryl Sulfate | 1.250% |
| Flavor | 1.2% |
| TOTAL | 100% |

¹⁾Tixosil 73; trademark and origin:
²⁾Tixosil 43; trademark and origin:

### Example 26

### Dicalcium Phosphate Based Toothpaste Formulation

A sufficient amount of microcapsule slurry M (corresponding to microcapsules A except that a menthol flavor is encapsulated) is weighed and mixed in the following composition (Table 35) to add the equivalent of 0.2% flavor.

TABLE 35
Toothpaste formulation

| Ingredients | Amount (% wt) |
|---|---|
| Sodium carboxymethyl cellulose | 1.2% |
| Flavor | 1.2% |
| DI/Purified Water | Q.S to Final Wt. |
| Sodium Lauryl Sulfate | 1.3% |
| Glycerine | 20.0% |
| Sodium Saccharin | 0.2% |
| Dicalcium phosphate dihydrate | 36.0% |
| Methylparaben | 0.2% |
| Silica¹⁾ | 3.0% |
| TOTAL | 100% |

¹⁾Aerosil ® 200; trademark and origin:

### Example 27

### Mouthwash Alcohol Free Formulation

A sufficient amount of microcapsule slurry M (corresponding to microcapsules A except that a menthol flavor is encapsulated) is weighed and mixed in the following composition (Table 36) to add the equivalent of 0.2% flavor.

TABLE 36
Mouthwash formulation

| Ingredients | Amount (% wt) |
|---|---|
| Propylene Glycol | 10% |
| Flavor | 0.240% |
| DI/Purified Water | Q.S to Final Wt. |
| Poloxamer 407 NF | 0.240% |
| Sodium Lauryl Sulfate | 0.040% |
| Sorbitol 70% Solution | 10.0% |
| Sodium Saccharin | 0.030% |
| Glycerine | 3.0% |
| Sodium Benzoate | 0.100% |
| Sucralose | 0.020% |
| Benzoic Acid | 0.050% |
| TOTAL | 100% |

### Example 28

### Mouthwash Formulation

A sufficient amount of microcapsule slurry M (corresponding to microcapsules A except that a menthol flavor is encapsulated) is weighed and mixed in the following composition (Table 37) to add the equivalent of 0.2% flavor.

TABLE 37
Mouthwash formulation

| Ingredients | Amount (% wt) |
|---|---|
| Ethyl Alcohol 190 Proof | 15.0% |
| Flavor | 0.240% |
| DI/Purified Water | Q.S to Final Wt. |
| Poloxamer 407 NF | 0.240% |
| Sodium Lauryl Sulfate | 0.040% |
| Sorbitol 70% Solution | 10.0% |
| Sodium Saccharin | 0.030% |
| Glycerine | 3.0% |
| Sodium Benzoate | 0.100% |
| Sucralose | 0.020% |
| Benzoic Acid | 0.050% |
| TOTAL | 100% |

1. A process for preparing a polyamide core-shell microcapsule slurry comprising the following steps:
a) Dissolving at least one acyl chloride and at least one stabilizer in a hydrophobic material to form an oil phase;
b) Dispersing the oil phase obtained in step a) into a water phase comprising optionally an amino compound A or a base to form an oil-in-water emulsion;
c) Performing a curing step to form polyamide microcapsules in the form of a slurry;
wherein at least an amino compound B is added in the water phase before the formation of the oil-in-water emulsion and/or in the oil-in-water emulsion obtained after step b).

2. The process according to claim 1, further comprising the step:
following step b) adding to the oil-in-water emulsion obtained in step b) the amino compound B.

3. The process according to claim 1, wherein the acyl chloride is chosen from the group consisting of benzene-1,3,5-tricarbonyl chloride, benzene-1,2,4-tricarbonyl trichloride, benzene-1,2,4,5-tetracarbonyl tetrachloride, cyclohexane-1,3,5-tricarbonyl trichloride, isophthalyol dichloride, diglycolyl dichloride, succinic dichloride, and mixtures thereof.

4. The process according to claim 1, wherein the water phase comprises a base chosen from the group consisting of sodium carbonate, sodium bicarbonate, sodium hydroxide, and mixtures thereof.

5. The process according to claim 1, wherein the water phase comprises the amino compound A chosen from the group consisting of L-Lysine, L-Lysine ethyl ester, guanidine carbonate, chitosan, 3-aminopropyltriethoxysilane and mixtures thereof.

6. The process according to claim 5, wherein the amino compound A is L-Lysine.

7. The process according to claim 1, wherein the amino compound B is chosen from the group consisting of cystamine, cystamine hydrochloride, cystine, cystine hydrochloride, cystine dialkyl ester, cystine dialkyl ester hydrochloride, a xylylene diamine, 1,2-diaminocyclohexane, 1,4-diaminocyclohexane, L-lysine, L-Lysine ethyl ester, polyetheramines, ethylene diamine, diethylene triamine, spermine, spermidine, polyamidoamine (PAMAM), guanidine carbonate, chitosan, tris-(2-aminoethyl)amine, 3-aminopropyltriethoxysilane, L-arginine and mixtures thereof.

8. The process according to claim 1, wherein the amino-compound A and the amino-compound B are different.

9. The process according to claim 1, wherein the stabilizer is chosen in the group consisting of gum Arabic, modified starch, polyvinyl alcohol, polyvinylpyrrolidone, carboxymethylcellulose, anionic polysaccharides, acrylamide copolymer, inorganic particles, proteins and mixtures thereof.

10. The process according to claim 9, wherein the stabilizer is a protein chosen from the group consisting of soy protein, rice protein, whey protein, white egg albumin, sodium caseinate, gelatin, bovine serum albumin, hydrolyzed soy protein, hydrolyzed sericin, pseudocollagen, silk protein, sericin powder, and mixtures thereof.

11. The process according to claim 1, wherein the molar ratio between the functional group NH₂ of the amino compound B and the functional group COCl of the acyl chloride is between 0.01 and 7.5.

12. The process according to claim 1, wherein the weight ratio between the acyl chloride and the hydrophobic material is between 0.01 and 0.09.

13. A polyamide core-shell microcapsule slurry obtainable by the process of claim 1.

14. A perfuming composition comprising:
(i) a microcapsule as defined in claim 13, wherein the hydrophobic material comprises a perfume,
(ii) At least one ingredient selected from the group consisting of a perfumery carrier and a perfumery base, and
(iii) Optionally at least one perfumery adjuvant.

15. A consumer product comprising:
a personal care active base, and
microcapsules as defined in claim 13,
wherein the consumer product is in the form of a personal care composition.

16. A consumer product comprising:
a home care or a fabric care active base, and
microcapsules as defined in claim 13,
wherein the consumer product is in the form of a home care or a fabric care composition.
